(19) United States
(12) Patent Application Publication
(10) Pub. No.: US 2009/0202440 A1
(43) Pub. Date: Aug. 13, 2009

(54) MODEL ANIMAL OF DENDRITIC CELL IMMUNORECEPTOR GENE KNOCKOUT DISEASE

(76) Inventors: Yoichiro Iwakura, Tokyo (JP); Shinobu Saijo, Tokyo (JP); Noriyuki Fujikado, Tokyo (JP)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/306,220

(22) PCT Filed: Jun. 26, 2007
(86) PCT No.: PCT/JP2007/000696

§ 371 Date: Feb. 23, 2009

(30) Foreign Application Priority Data
Jun. 28, 2006 (JP) 2006-177952
Feb. 9, 2007 (JP) 2007-030514

(51) Int. Cl. A61B 10/00; A01K 67/027; C12Q 1/18; C12Q 1/02; A61K 38/17; C07K 14/705; C12N 15/12; A61P 35/00; A61P 31/04; A61P 31/10; A61P 31/12; A61P 33/00

(52) U.S. Cl. 424/9.2; 800/11; 800/9; 435/32; 435/29; 514/12; 530/350; 536/23.5

(57) ABSTRACT

It is intended to disclose the function of dendritic cell immunoreceptor (DCIR) and clarify its role in the onset of autoimmune arthritis. It is also intended to provide a method of screening a substance which is useful in treating/preventing an autoimmune disease or osteoporosis. A nonhuman disease model animal characterized by having a partial or entire deficiency of a gene encoding the DCIR protein on the chromosome; a method of screening a substance which is useful in treating/preventing a DCIR-related disease, for example, an autoimmune disease such as arthropathy by using the nonhuman disease model animal as described above; and a remedy/preventive therefor.

### FIELD OF THE INVENTION

The present invention relates to a model animal of autoimmune diseases such as arthropathy, and bone diseases, characterized by having deficiency of a dendritic cell immunoreceptor gene (knockout (KO)). Furthermore, the invention relates to a screening method of a preventive/remedy using the model animal, and diagnosis and/or treatment of autoimmune diseases and bone diseases provided by the screening method.

### BACKGROUND ART

Rheumatoid arthritis (RA) is systemic chronic inflammatory disease mainly affecting synovial membrane at many joints. Inflammation of synovial membrane leads to damaged cartilage, bone erosion, deformed joint and loss of joint function. RA is an autoimmune disease characterized by infiltration of T-cells, B-cells, macrophage and neutrophil into surface of synovial membrane and fluid in cavities around joints.

The inventors have already established two types of model mice for RA by gene engineering of embryo. These are a human T cell leukemia virus type I transgenic (HTLV-I-Tg) mouse (patent document 1), and an IL-1 receptor antagonist defective (IL-1 Ra−/−) mouse (patent document 2), that spontaneously develop autoimmune and arthritis. The histopathological characteristics of the affected joints of these animals highly resemble that of human RA. The inventors conducted an exhaustive microarray analysis of gene expression on these two types of arthritis model mice, and found strong correlation in gene expression profile between a HTLV-I Tg mouse and an IL-1 Ra−/− mouse. In spite of apparent etiological difference between these mice, they showed similarity not only in histology but also in gene expression profile.

Particularly, the inventors newly found significant increase in expression of some genes on chromosome 6F2 site band of these RA model mice. The genes include calcium dependent (c type) lectin superfamily genes. C type lectine receptor (CLR) is a pattern recognition molecule that recognizes specific carbohydrate structure on the autoantigen or cell wall of pathogens, by extracellular carbohydrate recognition domain (CRD). CLRs including macrophage mannose receptor (MMR: CD206), dendritic cell specific ICAM3-grabbing non-integrin (DC-SIGN: CD209), L-SIGN and β-GR (Dectin-1) are-involved in recognition of various kinds of microbes such as virus, bacteria, fungus and some parasites. Interestingly, it is reported that signal transduction of CLRs are facilitated by that of Toll-like receptor (TLR), indicating possible cross-talk between these 2 signal transduction pathways. Also, CLRs regulate migration of dendritic cells and their interaction with lymphocytes. Other members of CLRs on NK cells such as human CD94/NKG2 and mouse Ly49 are involved in recognition of MHC class I to discern self from non-self. In short, CLRs play a role in natural immunity as well as acquired immunity. Some CLRs have signal transduction motifs such as immunoreceptor tyrosine-based inhibitory motif (ITIM) or immunoreceptor tyrosine-based activating motif (ITAM) in cytoplasm, which may regulate expression of function of dendritic cells.

Dendritic cells (DCs) are one of major antigen presentation cells, playing an essential role in regulation of immune system. Recently, expression of some CLRs on the surface of dendritic cells has been identified and characterization was made. MMR (CD206) and DEC-205 (CD205) belonging to type I CLR have plural calcium dependent extracellular carbohydrate recognition domains (CRDs) at its N-terminal. The second family of CLRs expressed on dendritic cells is type II protein having single CRD on its C-terminal, including DC-SIGN (CD209), Langerin (CD207), CLEC-1, Dectin-1 (β-GR), Dectin-2, DLEC and DCIR.

Among them, DCIR, that is also referred to as LLIR, is a type II membrane protein expressed mainly on dendritic cells of human and mouse. This molecule has single CRD in its extracellular domain, and a consensus ITIM domain in its intracellular domain. Since ITIM transmits inhibitory signal in the cell, it is suggested that mouse DCIR might serve as an inhibitory receptor to regulate function of dendritic cells. However, in vivo evidence of DCIR function has not been reported as of yet.

Patent Document 1: Japanese published unexamined application 6-38652

Patent Document 2: Japanese published unexamined application 2000-209980

### SUMMARY OF THE INVENTION

### Problems to be Solved by the Invention

The present invention was made on the inventors' findings about dendritic cell immunoreceptor (DCIR) of which expression is increased in the two types of mouse models established for rheumatoid arthritis (RA), one of autoimmune arthritis. The purposes of the invention are to provide a DCIR defective (KO) mouse (DCIR−/− mouse) to elucidate the role of DCIR in development of autoimmune diseases including arthritis, to demonstrate the usefulness of the DCIR−/− mouse as a disease model animal, to provide a method to screen a preventive/remedy for autoimmune diseases using the DCIR−/− mouse, and to provide a preventive/remedy for the autoimmune diseases using the screening method.

### Means to Solve the Problem

The objectives of the inventions are to:

(1) provide a nonhuman disease model animal characterized by having a partial or entire deficiency of a gene coding dendritic cell immunoreceptor (DCIR) protein on the chromosome; and
(2) provide a screening method of a substance that suppresses or facilitates differentiation and proliferation of bone-marrow stem cells derived cells, or a substance that suppresses development of arthritis, and to provide a preventive/remedy for autoimmune diseases containing compound or salt thereof identified by the screening method.

Further objectives of the invention are to:

(3) provide a preventive/remedy for autoimmune diseases comprising a substance that facilitates activity of DCIR protein or expression of gene coding DCIR protein, and to provide a diagnostic agent for autoimmune diseases comprising an entire or partial gene coding DCIR protein; and,
(4) provide a kit for screening a preventive/remedy for autoimmune diseases comprising a peptide having amino acid sequence identical or substantially identical to DCIR protein, a partial peptide or a salt thereof, and a diagnostic kit for autoimmune disease comprising a partial or entire DNA having base sequence identical or substantially identical to the gene coding DCIR protein.

### EFFECTS OF THE INVENTION

The DCIR defective mouse (KO mouse, hereinafter referred to as DCIR−/− mouse) according to the invention shows elevated sensitivity to collagen induced arthritis (CIA), thus, it is useful as a disease model animal of autoimmune diseases including arthritis. Also, an aged DCIR−/− mouse spontaneously develops autoimmune disease-like pathology such as inflammation at insertion of tendon or ligament to bone (enthesitis) or sialadenitis, which indicates regulation of immune system by DCIR. Thus, the disease model mouse according to the invention is markedly useful as an autoimmune diseases model mouse, the diseases including RA, ankylosing spondylitis (AS), diffused idiopathic skeletal hyperostosis (DISH), and Sjogren syndrome (SS).

Furthermore, the disease model animal according to the invention shows increased calcification at heel joint and decreased bone mass at femur compared to wild type animal. Thus, the mouse can be used as a model animal of bone diseases.

Also, the DCIR−/− mouse according to the invention shows increased anti-tumor immunity. Since DCIR plays a role in inhibitory function of dendritic cells, DCIR protein or equivalent thereof may be used as an activating agent in immunotherapy of cancer.

The model animal according to the invention allows analysis of physiological function and autoimmune disease inducing function of DCIR. Also, it can be used to elucidate mechanism of bone and cartilage diseases such as RA, AS, DISH or osteoporosis and to develop effective method and drug for treatment and diagnosis.

### BRIEF DESCRIPTION OF DRAWINGS

FIG. 1a is a graph that shows expression of DCIR mRNA in joints of an IL-1Ra−/− mouse and a HTL V-T-Tg mouse with arthritis by microarray analysis.

FIG. 1b shows expression of DCIR mRNA by northern blotting.

FIG. 2a is a schematic view that shows structures of DCIR locus on mouse DNA (wild type allele), DCIR targeting construct (targeting vector), and putative mutant DCIR gene (mutant allele), in which exons are indicated by boxes, that are used for production of a DCIR−/− mouse. Exon 1 and 2 of DCIR gene are substituted with neomycin resistance (Neo) gene. Diphtheria toxin (DT) gene is ligated to 3′ terminal of genome fragment for negative selection. External homologous region in the targeting allele is used as a genome probe for southern blotting. Southern blotting for screening used BanHI.

FIG. 2b is a drawing that shows the result of southern blot hybridization using a genome DNA fragment cleaved by BanHI of targeted ES clone and 5′ probe.

FIG. 2c is a drawing that shows the result of southern blot hybridization using a genome DNA fragment cleaved by EcoRI and 3′ probe.

FIG. 2d is a drawing that shows the result of southern blot hybridization using a genome DNA fragment cleaved by EcoRI and Neo probe.

FIG. 2e is a drawing that shows a result of southern blot hybridization to ensure proper targeting of DCIR region on a mouse DNA.

FIG. 2f is a drawing that shows expression of DCIR mRNA by northern blotting.

FIG. 3 is a drawing that shows characteristics of lymphocyte in a DCIR−/− mouse. This is a result of flow cytometry of thymus cells and spleen cells of a wild type (WT) mouse and a DCIR−/− mouse. For thymus cells and spleen cells, expression of CD4 and CD8 was analyzed, and for spleen cells, expression of CD3 and B220 was analyzed as well.

FIGS. 4a and 4b are drawings that show proliferation of T-cells dependent on dendritic cells.

FIG. 4a is a graph that shows incorporation of tritium thymidine by T cells derived from a spleen of a WT mouse when the T cells are co-cultured with dendritic cells derived from a WT mouse (WT) or a DCIR−/− mouse (KO), under presence of antigen nonspecific stimulus (anti CD3 antibody).

FIG. 4b is a graph that shows incorporation of tritium thymidine by T cells derived from a BALB/cA mouse (H-2^{d}) when the T cells are co-cultured with dendritic cells derived from a WT mouse (WT) or a DCIR−/− mouse (KO) with 129/Sv×C57BL/6) F1 background.

FIGS. 5a and 5b are graphs that show elevated production of autoantibody in DCIR−/− mice. The serum antibody level of DCIR−/− mice (n=19) and WT mice litter (n=19) at 12 months of age was determined by ELISA.

FIG. 5a shows total IgM, IgG and IgE levels.

FIG. 5b shows autoimmune antibodies level, particularly, it shows levels of antinuclear antibody (ANA), rheumatoid factor (RF IgM and RF IgG) and anti-IIC antibody. The graph illustrates mean and SEM. *p<0.05, **p<0.01

FIGS. 6a to 6d are drawings that show accumulation of dendritic cells and activation of T cells in aged DCIR−/− mice.

FIG. 6a is a drawing that shows stained CD11c and I-A^{b} of lymph node cells. The figures in the drawing shows rate of cells in a specific gate while the histogram at the bottom shows CD11c positive clusters in lymph node cells.

FIG. 6b is a graph that shows the result of flow cytometry to determine rate of CD11c positive cells in the lymph node cells of mice at 4 and 12 or above months of age. The graph illustrates mean and SEM. *p<0.05.

FIG. 6c is a drawing that shows stained CD4 and CD62L or CD44 on lymph node cells derived from a WT mouse and a DCIR−/− mouse at 12 months of age.

FIG. 6d shows a drawing that shows stained CD8 and CD62L or CD44.

FIGS. 7a and 7b are graphs that show aggravation of CIA in DCIR−/− mice. Chicken IIC was suspended with CFA, and injected at a plurality of percutaneous sites on the mouse tail. Development of CIA was observed without additional immunization. FIG. 7a shows incidence and 7b shows severity of CIA. The graphs illustrate mean and SEM of the pooled data of independent two tests. For statistical analysis, c² test is used for the test shown in FIG. 7a while Mann-Whitney U test is used for the test shown in FIG. 7b, with statistical significance of p<0.05.

FIGS. 8a to 8d are drawings that show elevated immune response in DCIR−/−mice when CIA was induced.

FIG. 8a is a graph that shows serum levels of IgM and IgG in a DCIR−/− mouse after IIC/CFA immunization.

FIG. 8b is a graph that shows levels of IIC-specific IgG subclasses. Serum of WT mice (WT, n=12) and DCIR−/− mice (KO, n=10) was collected before immunization (Pre) and 3 weeks after IIC/CFA immunization (3W), and level of IIC specific antibody was determined for each IgG subclass by ELISA.

FIG. 8c is a graph that shows proliferation response of IIC specific T cells. Lymph node cells were collected from WT mice (WT, n=9) and DCIR−/− mice (KO, n=9) 1 week after IIC/CFA immunization, cultured for 72 h under absence (Med) or presence (CII) of 100 mg/ml denatured chicken IIC to determine incorporation of tritium thymidine. The graph shows the pooled data of three independent experiments.

FIG. 8d shows a graph that shows cytokine production from lymph node cells. Levels of IFN-g, IL-4 and IL-10 were determined by ELISA in supernatant of lymph node cell culture of WT mice (WT, n=5) and DCIR−/− mice (KO, n=5) in the system shown in FIG. 8c. The graph shows mean and SEM. *P<0.05, **p<0.005.

FIGS. 9a and 9b are drawings that show elevated proliferation of dendritic cells in IIC/CFA immunized DCIR−/− mice. Lymph node cells were collected from WT mice (WT, n=3) and DCIR−/− mice (KO, n=3) 1 week after IIC/CFA immunization, and cultured for 72 h under absence (Med) or presence (CII) of 100 mg/ml denatured chicken IIC.

FIG. 9a is a graph that shows the result of FACS analysis of stained CD11c. The graph shows mean and SEM. *p<0.05, **p<0.01

FIG. 9b is a drawing that shows the result of flow cytometry of lymph node cells which were cultured for 72 h under absence (Med) or presence (CII) of denatured chicken IIC, then CD11c and I-A^{b}, CD80 or CD86 was double stained. The figures in the drawing show percentage of the cells in the gate.

FIGS. 10a to 10d are drawing that show elevated response of DCIR defective bone marrow cells (DCIR−/− BMC) to GM-CSF.

FIG. 10a is a graph that shows counts of cells derived from nonadhesive bone marrow determined at day 8 and day 10. The bone marrow cells derived from WT mice (WT) and DCIR−/− mice (KO) were cultured with GM-CSF added (n=4). *p<0.05, **p<0.01

FIG. 10b is a drawing that shows the result of flow cytometry of stained CD11c and I-A^{b} derived from nonadhesive bone marrow derived cells at day 8 of culture. The putative bone marrow derived dendritic cells are shown by the gate, and the figures show percentage of these cells to total cells.

FIG. 10c is a drawing that shows the result of flow cytometry of stained I-A^{b}, CD80 or CD86 of CD11c positive cells of bone marrow derived dendritic cells. The bone marrow derived dendritic cells were cultured for 48 h under absence (shaded histogram) or presence (open histogram) of LPS, and flow cytometry was conducted at day 10 of culture.

FIG. 10d is a drawing that shows STAT5 and phosphorylated STAT5 in bone marrow cells detected by anti-STAT5 antibody and anti-phosphorylated STAT5 antibody. Bone marrow cells were treated with GM-CSF at concentration shown in the figure for 20 minutes to adjust total cell solution for electrophoresis.

FIG. 11 is an X-ray image that shows skeleton of a DCIR−/− mouse and that of a WT mouse in Example 11.

FIG. 12 is an X-ray image that shows ankylosis site (heel) of a DCIR−/− mouse and a WT mouse in Example 11.

FIG. 13 is a cross-sectional view that shows the Von-Kossa strained (left) and Toluidine blue stained (right) heel joint of a DCIR−/− mouse in Example 11.

FIG. 14 is a photo that shows pQCT analysis of femur of a DCIR−/− mouse.

FIG. 15 is a graph that shows bone density and bone mineral of DCIR−/− mice and WT mice determined by pQCT analysis. The bone density is mean of selected regions, while bone mineral is mean of selected regions with thickness of 1 mm.

FIG. 16a is a graph that shows size of tumor and 16b is a graph that shows incidence of tumor, over time when DCIR−/− mice and WT mice were injected with MethA. WT: n=12, KO: n=11, **<0.05, *<0.01

FIG. 17 is a graph that shows change in incidence in DCIR−/− mice and WT mice injected with BALB/3T3 APR-MUC1 clone16. n=8, *<0.05.

FIG. 18 is a graph that shows the cytotoxity determined by using BALB/3T3 APR-MUC1 clone 16. *<0.01

### PREFERRED EMBODIMENT OF THE INVENTION

Preferably, the disease model animal according to the invention uses a rodent, more preferably, it uses a mouse. Since the model animal according to the invention spontaneously develops autoimmune diseases such as arthritis, it is useful as a model animal of autoimmune diseases. The autoimmune diseases that can be studied using the model animal according to the invention include rheumatoid arthritis (RA), ankylosing spondylitis (AS), Sjogren syndrome (SS) and diffused idiopathic skeletal hyperostosis (DISH), but not limited thereto. The mouse also can be used as a model animal of metabolic bone diseases such as osteoporosis, as well.

The screening method according to the invention comprises steps of administering a test substance to the disease model animal as above or exposing the tissue, organ or cell derived from the animal to the test substance, determining and evaluating the differentiation and proliferation of bone marrow stem cell derived cells in the animal or tissue, organ or cells thereof, and identifying a substance that facilitates or suppresses differentiation and proliferation of bone marrow stem cell derived cells. Alternatively, the method comprises steps of administering the test substance to the disease model animal, determining and evaluating incidence and score of collagen induced arthritis, and identifying a substance to suppress development of arthritis.

Preferably, the screening method according to the invention further comprises a step of comparing the result of determination and evaluation of the disease model animal with that of a wild type animal.

The screening method as above can identify a substance effective for prevention and treatment of autoimmune diseases including RA, AS, psoriatic arthritis (PsA), SS and DISH, and osteoporosis.

As mentioned above, the disease model animal according to the invention (DCIR−/− mouse) spontaneously develops or aggravates autoimmune diseases. This finding indicates that DCIR protein is effective in prevention and treatment of the disease. Therefore, the invention provides a preventive/remedy for autoimmune diseases or osteoporosis, the preventive/remedy comprising a substance that facilitates expression of gene coding DCIR protein.

Furthermore, since the DCIR protein defective animal develops autoimmune diseases, a gene coding DCIR protein or a partial sequence thereof is effective as a diagnostic agent of autoimmune diseases or osteoporosis. The invention provides a diagnostic agent of autoimmune diseases or osteoporosis, comprising such DNA or partial sequence thereof.

A peptide having an amino acid sequence identical or substantially identical to DCIR protein, a partial peptide or a salt thereof can screen a substance that facilitates activity of DCIR protein, that is, a preventive/remedy for autoimmune diseases or osteoporosis, by co-culturing the peptide, the partial peptide or the salt thereof with a candidate substance, and comparing the activity of DCIR protein under presence or absence of the candidate substance. The invention provides a kit for screening such a preventive/remedy.

A base sequence identical or substantially identical to the gene coding DCIR protein, or partial sequence thereof, can be used as a probe to determine presence or absence and degree of expression of DNA coding DCIR and to determine susceptibility of a subject to autoimmune diseases or osteoporosis. The invention provides a kit for determining such susceptibility.

The inventors clarified for the first time that DCIR is involved in development or aggravation of autoimmune diseases. DCIR is one of inhibitory regulators in immune system, playing a role to suppress immune response under physiologic condition. Therefore, by administering a peptide comprising an amino acid sequence identical or substantially identical to DCIR protein (soluble extracellular protein moiety, in particular), or a partial peptide or salt thereof, inhibitory signals in immune system may be inhibited, activating immune system. Therefore, the peptide, the partial peptide or salt thereof can be used as a remedy for various infections caused by various viruses, bacteria, fungi or protozoa.

Recently, in cancer immunotherapy, a method comprising making dendritic cells intake tumor cells or antigen in vitro, activating and maturing the dendritic cells and administering the cells to a patient attracts attention as the method may activate immune response of the patient. This method has another advantage of lower risk of adverse effect because it uses dendritic cells derived from the patient himself. Therefore, the peptide of DCIA protein or equivalent thereof may be useful as an activating agent for cancer immunotherapy.

### EXAMPLES

Next, the invention will be described in detail with reference to specific embodiment thereof, but the invention is not limited to these embodiments.

### Experiment Method

### Mouse:

Two mouse models, HTLV-I-Tg mice and IL-1 Ra−/− mice were used for gene expression profiling. HTLV-I-Tg mice were prepared by injecting LTR-env-pX-LTR region of HTLV-I genome to (C3H/Hen×C57BL/6J) F₁ embryos. The resulting mice were backcrossed for 20 generations to BALB/cA mice (CLEA Japan, Tokyo, Japan). The resulting mice spontaneously develop arthritis at 4 weeks of age, the incidence being 60% at 3 months of age, and 80% at 6 months of age. In the following examples, HTLV-I-Tg (TS) mice (female at 6 to 9 weeks of age) that developed severe arthritis (score 3) were used. IL-1Ra−/− mice prepared by homologous recombination were backcrossed for 8 generations to BALB/cA mice. These mice spontaneously develop arthritis at 5 weeks of age, the incidence being 80% at 8 weeks of age and 100% at 13 weeks of age. In the following examples, IL-1Ra−/− mice (KS) (male at 13 weeks of age) that developed severe arthritis (score 3) were used. Wild type litter mice (WT) were used as a control.

The severity of arthritis was scored from 0 to 3 based on the extent of redness and swelling of feet. Score 0=normal, Score 1=mild swelling in joint and/or redness in foot, Score 2=marked swelling of joint, Score 3=severe swelling and ankylosis of joint. All mice were bred under specific pathogen free (SPF) condition in a clean room of The Institute of Medical Science, The University of Tokyo. All experiments were conducted in conformity with ethical guidelines for animal experimentation and safety standard regarding to gene engineering.

Northern Blot Hybridization:

The joint tissue was immediately frozen in liquid nitrogen, and kept at −80° C. The frozen tissue was homogenized with physcotron (Microtech, Chiba, Japan). From the homogenate, total RNA was prepared using guanidine isothiocyanate phenol chloroform method, and poly (A)⁺RNA was purified using oligo (dT) cellulose column. For each sample, RNA derived from 4 to 5 mice was pooled. Poly (A)⁺RNA was electrophoresed in 1.3% denaturing agarose gel, and transferred to a nylon membrane (Gene Screen Plus, NEN Life Science, Boston, Mass., USA). Hybridization was conducted using ³²P-labeled DNA probe labeled with Megaprime DNA labeling system (Amersham, Arlington Heights, Ill., USA) and ³²P-dCTP (3,000 Ci/mmol: NEN Life Science, Boston, Mass., USA) at 42° C. over night. Radioactivity was determined by BAS-2000 system (Fuji Photo Film Co., Tokyo, Japan). Mouse DCIR probe was amplified from cDNA derived from joint of an HTLV-I-Tg mouse with arthritis, using PCR. The following PCR primers were used:

| | |
|---|---|
| 5′-CAT TTC CCT TAT CTC GCC CTG G-3′ | (SEQ NO: 4) |
| 5′-GCA GCA TGA ATG TCC AAG ATC C-3′ | (SEQ NO: 5) |

Preparation of DCIR−/− Mouse:

The sequence of genome DNA containing DCIR was obtained from mouse genome database provided by Celera Genomics (Rockville, Md., USA), and a 5′ homology region comprising a 5.5-kb fragment and a 3′ homology region comprising a 2.5-kb fragment were amplified from genome DNA derived from ES cells (E14.1) by PCR. Following sets of PCR primers were used:

5-arm:

(SEQ NO: 6)

5′-GAT TAA AAG CGG CCG CCA GAA TTC GTT TGA GAT CAG
GC-3′

(SEQ NO: 7)

5′-CTG GAT CCG TCA GAA GAG AGC CTT GTT CC-3′
3′-arm:

(SEQ NO: 8)

5′-CCA TCG ATG AAG AGA GGT TCC ACT CTA GC-3′

(SEQ NO: 9)

5′-TTA TCG ATG TCA ACT ACC TTT GCA TTG GG-3′

Targeting vector was prepared by substituting a genome fragment coding the first and second exons containing the intracellular domain containing ITIM and the transmembrane domain, with a fragment expressing neomycin resistance gene (Neo) under control of PGK1 promoter. For negative selection, a fragment expressing diphtheria toxin (DT) under control of MC1 promoter was ligated to 3′-terminal. The targeting vector was introduced to ES cells by electoporation and the resultant cells were selected by G418. 672 neomycin resistant ES clones were picked up, from which 660 clones were screened by southern blot hybridization using 5′ probe.

5′ probe used for the screening was amplified using the following primers:

| | |
|---|---|
| 5′-TAA CAC TGA GGG AAG ATG CTA C-3′ | (SEQ NO: 10) |
| 5′-TCT CAT TCT CAC TCT CAC TCT C-3′ | (SEQ NO: 11) |

96.2% of the clones were identified by southern blot hybridization, and 2 targeting ES clones were identified (Targeting efficiency: 0.3%). These targeting clones were confirmed by 3′-probe and Neo probe.

3′-probe was amplified using the following primers:

| | |
|---|---|
| 5′-AGC CAT GAT AAC AGA CCC-3′ | (SEQ NO: 12) |
| 5′-TGA TAT GGG GTC TGG TAC G-3′ | (SEQ NO: 13) |

Narl-Xbal fragment of neomycin resistant gene was used as Neo probe. Karyotyping showed that about 80% of the cells of both clones has normal 40 chromosomes. Chimera mice were prepared by aggregation method. A male chimera mouse was mated with a wild type C57BL/6J female mouse, and transmission to the germ cells was determined by fur color. Heterozygosis mice in terms of DCIR mutation were mated to prepare homozygosis mice. In the following examples, DCIR−/− mice and its litter mice with (129/Sv×C57BL/6) F₁ genetic background were used.

Genotyping of DCIR−/− mouse used the following PCR primers:

Common primer:

(SEQ NO: 14)

5′-AAG TGT CCC CTC TTG TAC TCT GTG-3
Wild type primer:

(SEQ NO: 15)

5′-CAA AAT TCT GTC AAG CGT AGA GGG G-3′
Mutant primer:

(SEQ NO: 16)

5′-CAT TAT ACG AAG TTA TCT CGA GTC GC-3′

The common primer and the wild type primer were used to detect wild type allele (1.3 kb) and the common primer and the mutant primer were used to detect mutant allele (0.9 kb).

Histopathology:

A WT mouse and a DCIR−/− mouse at 12 months of age were anesthetized with ether, and perfusion fixed with neutral buffer 10% formalin solution. Major organs and tissues including brain, heart, aorta, lung, bronchium, pancreas, liver, spleen, axillary lymph node, submaxillary gland and parotid gland, intestine, stomach, kidney and reproductive tract were excised for histopathologic observation. Joint tissues including foot, knee, wrist and thoracic vertebra were decalcified using 10% formic acid and paraffin embedded. For each tissue, 2 to 3 mm sections were prepared and stained with hematoxylin and eosin.

Flow Cytometry:

Cells were stained with following monoclonal antibodies (mAb) conjugated with FITC, PE or biotin, and used for flow cytometry. mAb of a hamster, a mouse or a rat specific to CD11c (HL3), I-Ab (AF6-120.1), CD3 (145-2C11), CD45R/B220 (RA3-6B2), CD4 (RM4-5), CD8 (53-6.7), CD62L (MEL-14) or CD44 (IM-7) were purchased from BD PharMingen (San Diego, Calif., USA). Rat mAb specific to CD80 (RMMP2), CD86 (RMMP1) of a mouse was purchased from Immunotech (Marseille, France). PE conjugated StreptAvidin (PharMingen) was used for secondary staining of biotin conjugated antibody. The surface of cells were stained using standard protocol, and analysed using FACSCalibur and CellQuest (Becton Dickenson, Franklin Lakes, N.J., USA) or FlowJo (Tree Star, Inc. Ashland, Oreg.) software.

Proliferation:

In order to investigate antigen specific response, Thy 1.2 positive T cells were prepared from a WT mouse, CD11c positive dendritic cells were purified from spleens of a WT mouse and a DCIR−/− mouse by magnetic cell sorting using CD90 (Thy1.2) microbeads or CD11c microbeads (MiltenyiBiotech, Bergisch Gladbach, Germany). Purified Thy1.2 positive T cells (2×10⁵ cells) and CD11c positive dendritic cells (2×10⁴ cells) were co-cultured for 3 days with anti-CD3 antibody (145-2C11: BD PharMingen), and left to incorporate tritium thymidine (0.25μ Ci/ml: Amersham, Buckinghamshire England) for 6 hours. Next, the cells were collected on a Micro 96 cell harvester (Skatron, Lier, Norway) glass fiber filter, and radioactivity of tritium thymidine was determined using Micro Beta (Pharmacia Biotech, Piscataway, N.J.).

In order to investigate allogeneic mixed leucocyte reaction, Thy1.2 positive T cells were purified from a BALB/c mouse by magnetic cell sorting using CD90 (Thy1.2) microbeads. Dendritic cells were induced from bone marrow cells of a WT mouse and a DCIR−/− mouse. The dendritic cells were treated with mitomycin C, co-cultured with Thy1.2 positive T cells for 3 days, and left to incorporate tritium thymidine for 6 hours to determine radioactivity thereof.

Collagen Induced Arthritis:

To complete Freund's adjuvant (Difco Laboratories Detroit, Mich.) added with 5 mg/ml heat killed M tuberculosis (H37RA: Difco), 1 mg/ml chicken II type collagen (IIC: Sigma-Aldrich, St. Louse, Mo.) was suspended, and the suspension was immunized to several subcutaneous sites of the tails of the mice. Arthritis was induced without additional immunization. Their joints were observed as to presence of swelling and redness for several days, and if any swelling or redness were observed, its severity was scored to 0 to 3. (Score 0=normal: Score 1=mild swelling of joint and/or redness on foot: score 2=marked swelling of joint: Score 3=severe swelling and ankylosis of joint). For histological evaluation of arthritis, the joint tissue was fixed in 10% neutral buffer formalin solution, decalcified with 5% formic acid, and paraffin-embedded. From tissues, 4 mm sections were prepared and stained with hematoxylin and eosin.

Determination of Antibody:

10 mg/ml chicken IIC for determining collagen specific antibody; rabbit anti mouse IgM (2 mg/ml: Zymed), IgG (8.7 mg/ml: Zymed) or IgE (2 mg/ml: PharmMingen) for determining serum antibody level; heat-denatured rabbit IgM, IgG (50 mg/ml: Zymed) or chicken IIC (20 mg/ml: Sigma) for determining autoantibody, were coated to Falcon 3912 Micro Test III flexible Assay plate (BD Bioscience. Oxnard, Calif.) overnight at 4° C. After washed with PBS, diluted serum samples were incubated for 1 hour at room temperature. After washed with PBS-Tween, alkaline phosphatase (AP) conjugated goat anti-mouse IgM, IgG, IgE, IgG1, IgG2a, IgG2b and IgG4 were incubated for 1 hour at room temperature, and AP activity was determined by ELISA microreader (MTP-120: Colona, Tokyo, Japan) using Substrate Phosphatase SIGMA104 (Sigma-Aldrich). For determining antinuclear antibody, diluted mouse serum was incubated on mouse nucleus antigen coating plate (Alpha Diagnostic, Inc., San Antonio, Tex.), reacted with horseradish peroxidase (HRP) conjugated goat anti mouse IgG, and HRP activity was determined using TMB substrate.

Collagen Specific Proliferation Reaction and Cytokine Production:

Lymph nodes were collected 7 days after IIC/CFA immunization to prepare single cell suspension. Lymph node cells were incubated under presence or absence of heat-denatured IIC for 3 days, left to incorporate tritium thymidine (0.25 mCi/ml) for 6 hours to determine tritium thymidine radioactivity. For determining cytokine production, culture supernatant was collected 3 days after collagen specific proliferation reaction, and IFN-γ, IL-4 and IL-10 levels were determined using BD OptiEIA ELISA Set (BD PharMingen).

Preparation of Bone Marrow Cell Derived Dendritic Cells

Bone marrow cell derived dendritic cells were prepared from bone marrow of femur and tibia. Briefly, 2×10⁵ cells/ml of bone marrow cells were cultured in 10% FCS-RPMI-1640 added with 20 ng/ml recombinant mouse GM-CSF (PeproTech, London, UK). At day 3, double dose of fresh medium containing 20 ng/ml GM-CSF was added, and at day 6 and 8, half of medium supernatant was collected for centrifugation. The cells were re-suspended in a fresh medium containing 10 ng/ml GM-CSF and returned to the original plate. Non-adhesive cells at day 10 were used as bone marrow cell derived dendritic cells. Furthermore, for complete maturation of bone marrow cell derived dendritic cells, non-adhesive cells at day 10 were collected, and re-suspended in a fresh medium containing 10 ng/ml GM-CSF and 1 mg/ml LPS (Sigma). These cells were incubated for additional 2 days, and used as matured dendritic cells.

Western Blotting:

Bone marrow cells were prepared from femur and tibia, and cultured overnight on nonserum RPMI-1640 medium added with 20 ng/ml recombinant mouse GM-CSF. The cells were washed and resuspended in nonserum medium without GM-CSF. Cells were left starved for 6 hours, treated with GM-CSF and phosphatase inhibitor cocktail II (Sigma) at various concentration. Total cell solution was analyzed by immuno blotting using anti-phosphorylated STAT 5 antibody (Cell Signaling, Denvers, Mass.) and anti STAT5 antibody (Santa Cruz Biotechnology, Inc., Santa Cruz, Calif.). Horseradish peroxidase (HRP) conjugated anti rabbit IgG (Cell Signaling) was used as detection antibody. Luminescent signal was detected using ECL plus detection solution and Typhoon 9000 (Amersham Biosciences, Buckinghamshire, UK).

Statistics:

For statistical analysis, Student t-test was used except followings: X2 test was used for incidence of CIA experiment; Mann-Whitney U-test was used for severity, and incidence of hisopathological abnormality.

### Example 1

### Identification of DCIR as RA Related Gene

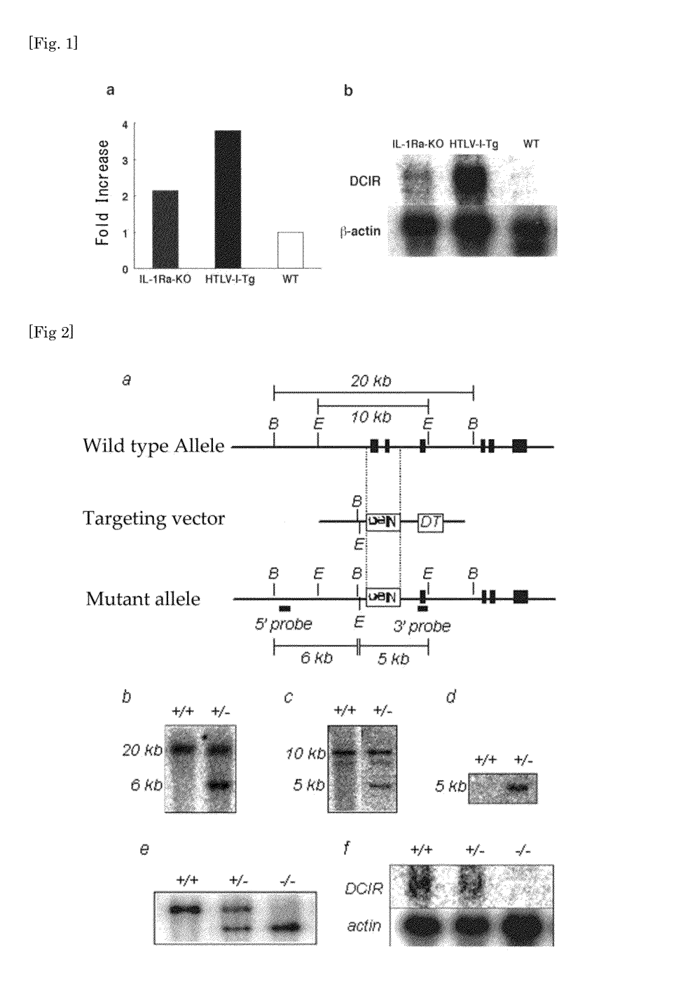

Gene expression profile was compared between joints of two types of model mouse (HTLV-I Tg and IL-1Ra−/− mouse) and that of a normal mouse to identity gene showing elevated expression in joints with arthritis. Microarray analysis revealed mRNA expression of DCIR gene was 2.2 times in an IL-1 Ra−/− mouse, and 3.8 times in an HTLV-I Tg mouse, compared to that in a WT mouse (FIG. 1a). This elevated expression was also confirmed by northern blot hybridization (FIG. 1b).

### Example 2

### Preparation of DCIR−/− Mouse

A DCIR−/− mouse was prepared by conventional gene targeting method.

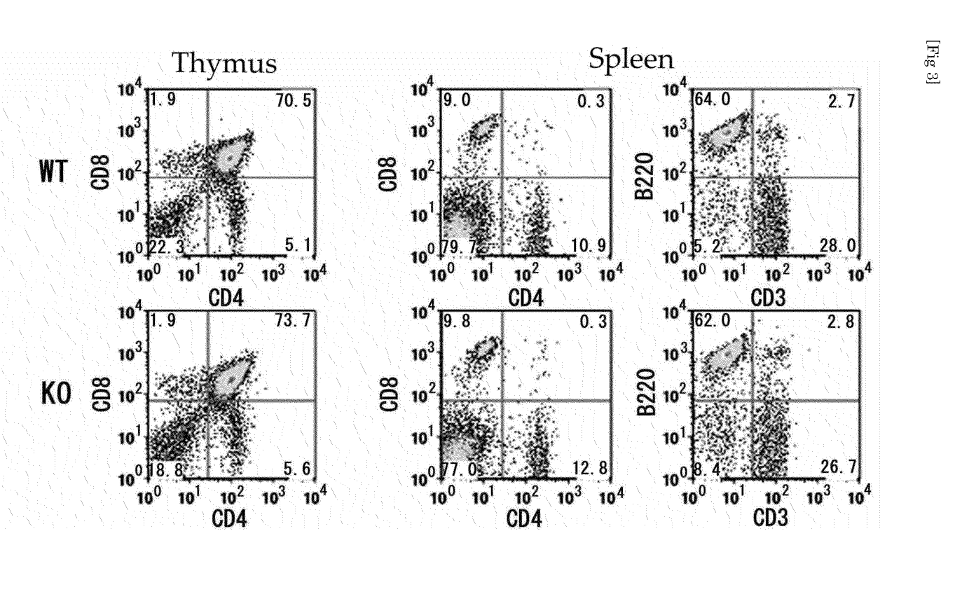

Exons 1 and 2 of DCIR gene were substituted with neomycin resistant gene to delete genome sequence that codes major part of cytoplasmic domain including ITIM and transmembrane domain (FIG. 2a). Targeting ES clone was screened by southern blot hybridization using 5′ probe (FIG. 2b), and targeting allele was identified by 3′ probe (FIG. 2c) and Neo probe (FIG. 2d). Targeting of DCIR domain in a mouse was confirmed by genome southern blotting (FIG. 2e). DCIR−/− mice was prepared by mating DCR+/−mice, and it was confirmed that the resultant DCIR−/− mice lacked expression of DCIR mRNA in the spleen by northern blot hybridization (FIG. 2f).

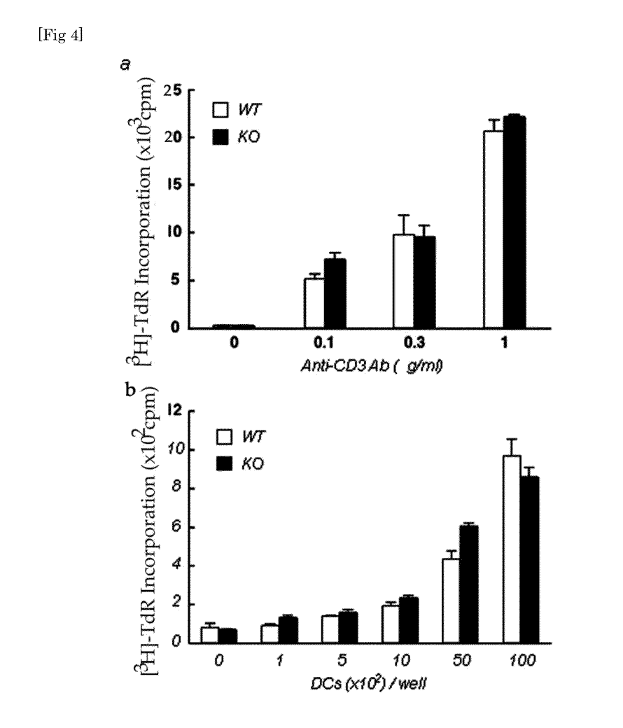

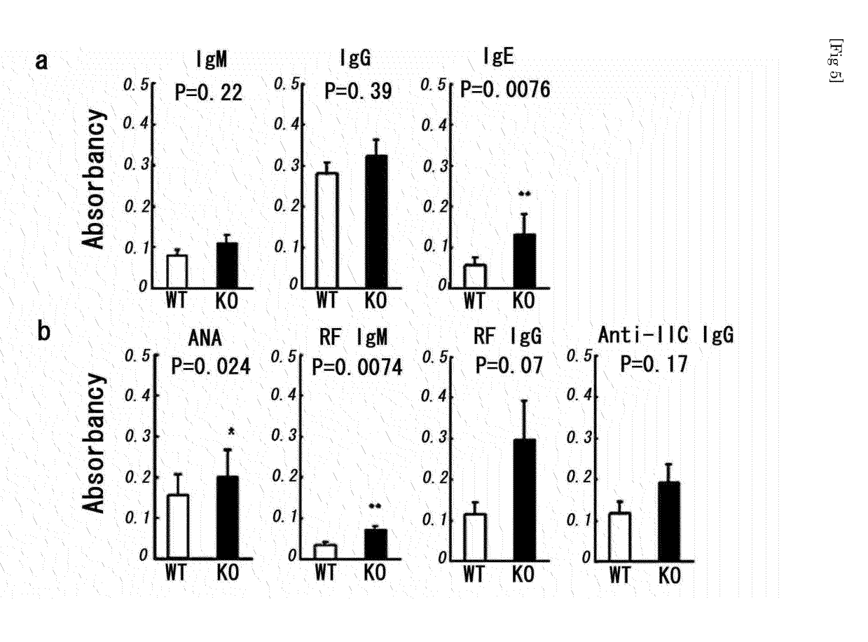

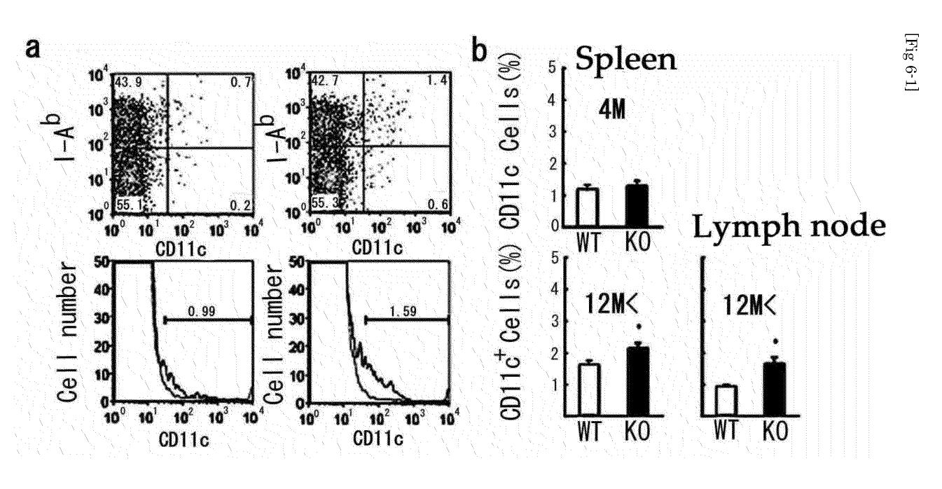

DCIR−/− mice were fertile, and their offspring showed Mendelian inheritance. Young DCIR−/− mice showed no aberrant phenotype under specific pathogen free condition. No abnormality was observed in spleen and thymus cells of DCIR−/− mice (FIG. 3). In order to determine effect of DCIR deficiency on T cell response, proliferation and allogeneic mixed lymphocyte reaction (MLR) of T cells induced by stimulation by anti CD3 antibody was determined under presence of dendritic cells. The result showed no significant difference between DCIR defective dendritic cells and wild type dendritic cells (FIG. 4).

### Example 3

### Spontaneous Development of Autoimmune Diseases in Aged DCIR−/− Mice

Histology of DCIR−/− mice revealed accumulation of lymphocytes in salivary gland interstitium, infiltration of monocytes into epithelium of minor conduit and associated sialadenitis characterized by damage to minor conduit, in submaxillary glands of 7 in 10 DCIR−/− mice at 6 to 12 months of age (Table 1).

At 4 months of age, 11% of DCIR−/− mice showed abnormal joints in their hind legs. Initially, the mice showed arthritis with redness and swelling in a plurality of joints, and by 6 months of age, 28% of mice developed arthritis. Incidence and severity of arthritis were worsened over time, bringing about deformation and ankylosis of joints. The symptoms were marked in joints in hind legs. Incidence showed sex difference: the percentage of mice that developed symptom was 44% in the male mice while 6% in the female mice at 12 months of age. Histological observation of joints of DCIR−/− mice revealed infiltration of inflammatory cells to insertion of tendon and ligament to bone, and destruction of bone by invading granulated tissue. Enthesitis was present in 9 in 23 DCIR−/− mice observed (Table 1). These findings indicate that deficiency of DCIR gene induces autoimmune, and spontaneous development of delayed arthritis and sialadenitis.

TABLE 1
Incidence of histopathological abnormality in aged DCIR-/- mouse

| | | Number of mouse with | | | | | |
|---|---|---|---|---|---|---|---|
| | | symptom/total mouse (%) | | | | | |
| Symptom | Genotype | Male | | Female | | Total | |
| Sialadenitis | Wild type | 0/3 | (0) | 0/2 | (0) | 0/5 | (0) |
| | DCIR-/- | 4/6 | (67)* | 3/4 | (75)* | 7/10 | (70)** |
| Enthesitis | Wild type | 0/5 | (0) | — | | 0/5 | (0) |
| | DCIR-/- | 7/17 | (41)* | 2/6 | (33)* | 9/23 | (39)* |

The table shows number of mice and incidence (figures in parenthesis) of sialadenitis and enthesitis in mice at 6 to 12 months of age
*P < 0.05,
**p < 0.01 (Mann-Whitney U test)

### Example 4

### Spontaneous Development of Autoimmune in DCIR−/− Mice

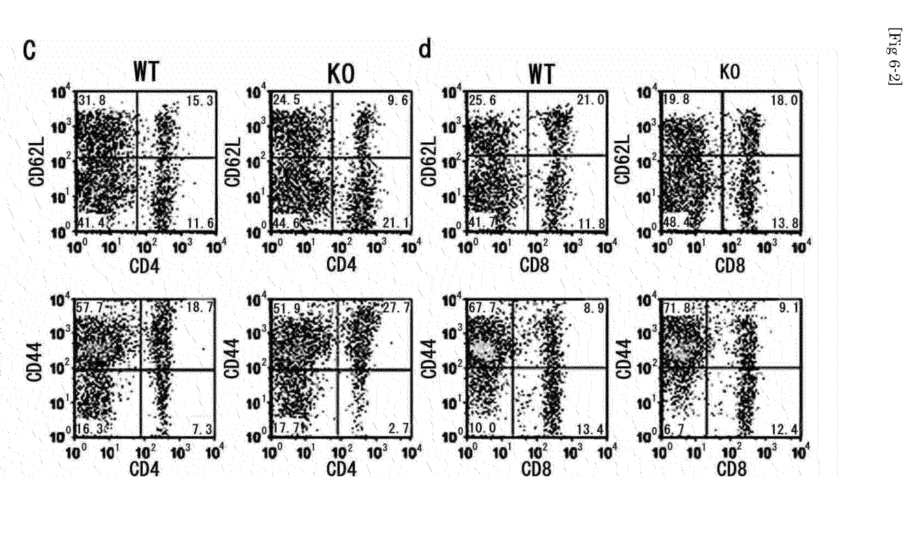

Since it was indicated that DCIR deficiency influenced development of autoimmune, production of autoantibody in DCIR−/− mice was determined by ELISA. Serum levels of IgM, IgG, IgE and production of autoantibodies such as antinuclear antibody (ANA), rheumatoid factor (RF: IgM and IgG types), and anti-IIC IgG were determined (FIG. 5). There was an increasing tendency in serum immune globulin level of DCIR−/− mice at 12 months of age, without significant difference except IgE level. Aged DCIR−/− mice showed increased production of autoantibodies such as antinuclear antibody (ANA), rheumatoid factor (RF) and anti-IIC antibody, compared to WT mice. The findings indicate that DCIR deficiency relates to production of autoantibodies.

### Example 5

### Proliferation of Dendritic Cells and Activation of CD4 Positive T Cells in a DCIR−/− mouse

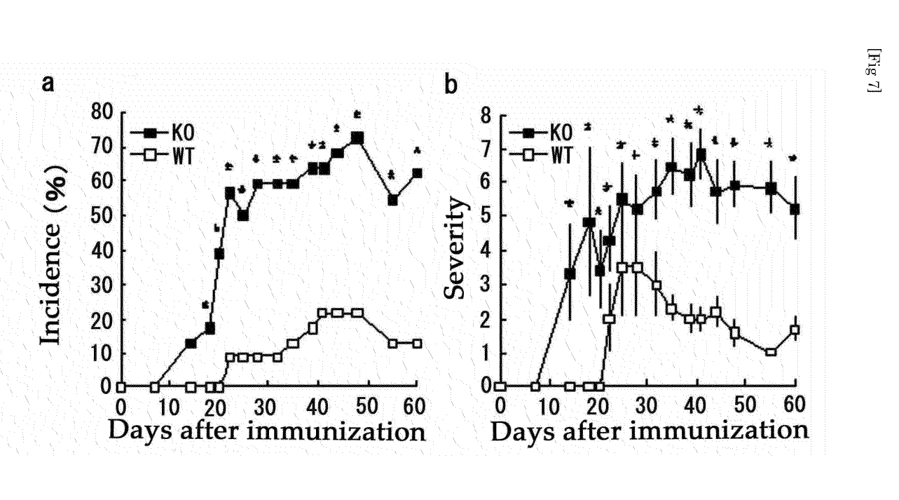

The ratio of CD11c positive cells that expressed DCIR was determined in a DCIR−/− mouse. A young mouse showed no abnormality in the ratio under physiological condition, but a mouse at 12 or more months of age showed marked increase in the ratio. Most of CD11c positive cells expressed I-Ab. CD4 positive T cells of a DCIR−/− mouse had lower expression of CD62L that is a characteristic of activated T cells, but higher expression of CD44. CD8 positive T cells showed no marked change (FIG. 6).

### Example 6

### Aggravation of Collagen Induced Arthritis in a DCIR−/− Mouse

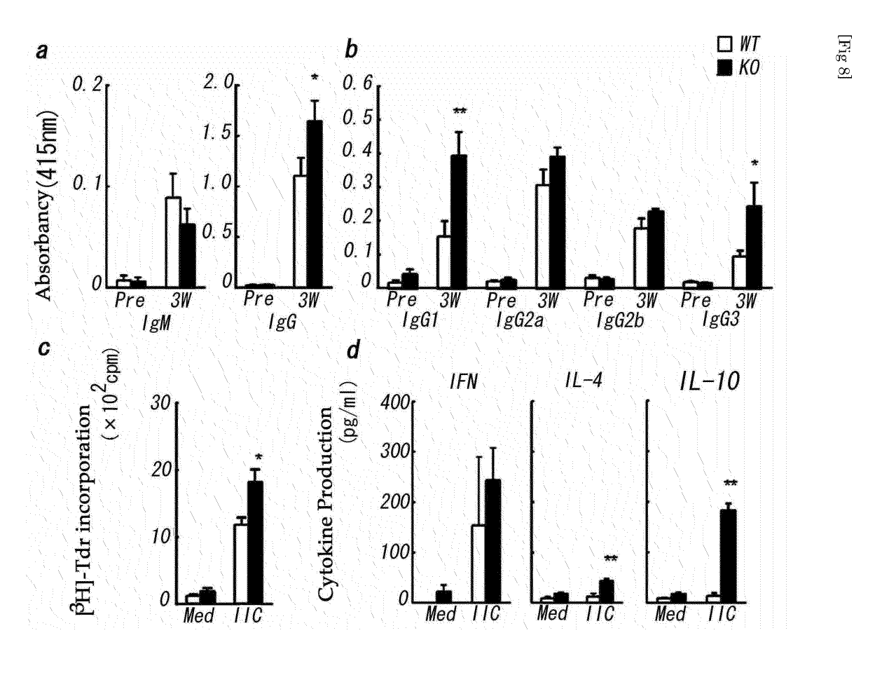

Since expression of DCIR was increased in the RA model mouse according to the invention, the effect of DCIR deficiency on development of collagen induced arthritis (CIA) was tested. CIA was induced by immunizing 250 mg of complete Freund's adjuvant added with heat-killed Mycobacterium tuberculosis, and 100 mg of chicken type II collagen (IIC) to young DCIR−/− mice with (129.Sv×C57BL/6) F₁ hybrid background. In a conventional protocol, additional immunization with IIC/CFA is to be conducted at day 21 after the first immunization. However, in this experiment, 20 days after first immunization, 0% of litter and 39% of DCIR−/− mice developed symptom (FIG. 7). When additional immunization is used, it might be difficult to observe increase in incidence in DCIR−/− mice, so that the observation was continued without additional immunization. As shown in FIG. 7, the incidence of arthritis in DCIR−/− mice was markedly higher than that in the litter as control. Also, the DCIR−/− mice developed significantly severer symptom compared to WT mice. The incidence and severity in the groups are accumulated data of two independent experiments.

Histology of CIA in the joints of control mice revealed minimum cell infiltration with no proliferated synovial membrane or destroyed bone. On the other hand, DCIR−/− mice developed typical arthritis with infiltration of inflammatory cells, proliferation of synovial membrane and destruction of bone even under this mild immunization. The findings indicate that DCIR suppresses development of CIA.

### Example 7

### Increased Production of Antigen Specific IgG1 and IgG3 Antibodies in DCIR−/+ Mice

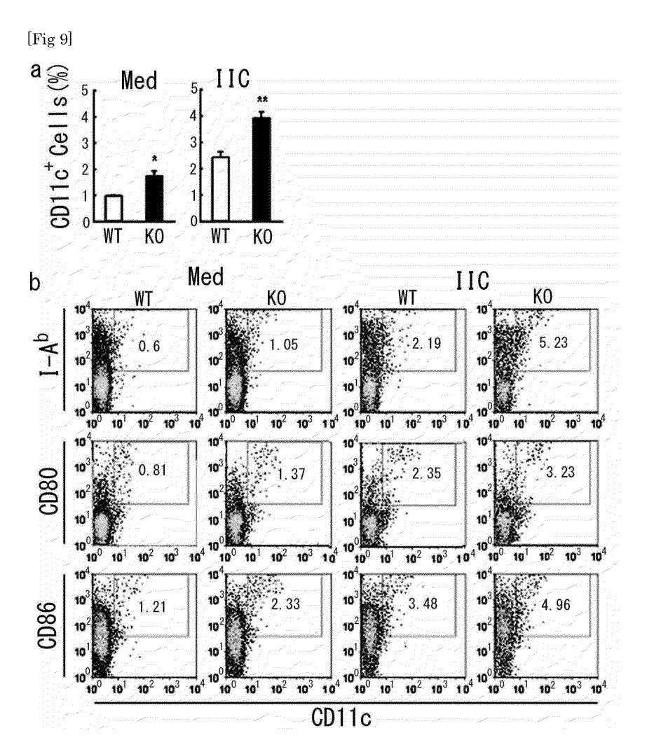

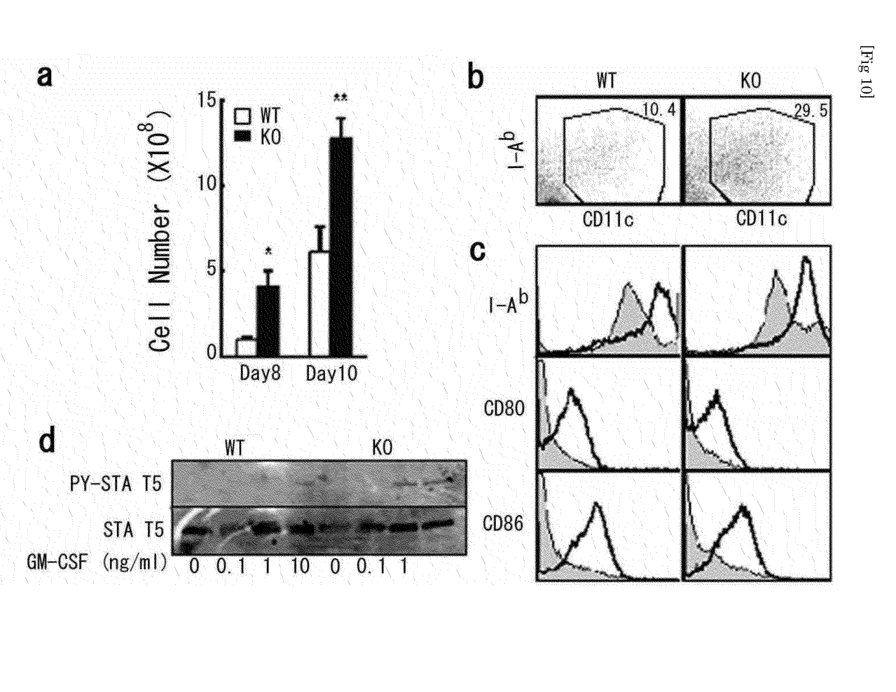

Next, production of IIC specific antibody in DCIR−/− mice immunized with IIC was determined. It is known that level of antibody against IIC is well-correlated to development of arthritis. As shown in FIGS. 8a and 8b, levels of IgG, IgG1 and IgG3 specific to IIC were significantly higher in DCIR−/− mice than in the wild type litters. On the other hand, levels of IgM, IgG2a and IgG2b in DCIR−/− mice showed no abnormality. These findings indicate involvement of DCIR in production of IgG1 and IgG3 subclass antibodies specific to antigen in CIA.

### Example 8

### Elevated Antigen Specific Proliferation of T Cells in DCIR−/− Mice

Antigen specific proliferation of T cells derived from DCIR−/− mice and control litters was tested. Reaction of T cells to IIC was determined 7 days after initial immunization with chicken IIC/CFA. Antigen specific proliferation of T cells was markedly higher in DCIR−/− mice than in WT mice (FIG. 8c), indicating that priming of T cells was facilitated in DCIR−/− mice. Production of IFN-γ from lymph node cells after stimulation with IIC was observed similarly in DCIR−/− mice and control mice. On the other hand, levels of IL-4 and IL-10 were significantly higher in DCIR defective lymph node cells (FIG. 8d). These findings indicate that production of cytokine from Th2 was selectively facilitated in a DCIR−/− mouse, consistent with observation of facilitated production of IIC specific IgG1 and IgG3 subclass antibodies.

### Example 9

Determination of content and activity of DC after IIC/CFA immunization revealed marked increase in the ratio of CD11c positive cells in lymph node of DCIR−/− mice. Furthermore, the ratio of activated dendritic cells that expresses I-A^{b}, CD80 and CD86 was markedly higher in DCIR−/− mice than in WT mice. These findings demonstrate that differentiation and activation of dendritic cells in a DCIR−/− mouse is facilitated by IIC/CFA immunization.

### Example 10

### Elevated Reaction of DCIR Defective Bone Marrow Cells (BMC) to GM-CSF

Since DCIR expresses mainly on dendritic cells, differentiation of DCIR defective bone marrow cells to dendritic cells was determined. When DCIR defective bone marrow cells were cultured with GM-CSF, differentiation of non-adhesive bone marrow derived cells was significantly higher in DCIR defective bone marrow cells (FIG. 10a). Flow cytometry of non-adhesive cells at day 8 of incubation showed marked increase of CD11c positive bone marrow cells derived dendritic cells (BMDC) in the culture of DCIR defective bone marrow cells (FIG. 10b). Most of these CD11c positive cells expressed I-A^{b}, without expression of activated markers CD80 and CD86. When BMDC at day 10 of culture was subject to LPS pulse for 48 hours, the dendritic cells derived from a DCIR−/− mouse and a WT mouse showed marked expression of I-A^{b}, CD80 and CD86, indicating similar level of maturation of both types of cells (FIG. 10c).

Next, the phosphorylation of STAT5 induced by GM-CSF stimulus in bone marrow cells derived from a DCIR−/− mouse was analyzed. As shown in FIG. 10d, activation of STAT 5 induced by GM-CSF was markedly increased in DCIR defective bone marrow cells, consistent with elevated reactivity of DCIR defective bone marrow cells to GM-CSF. Therefore, elevated differentiation of DCIR defective bone marrow cells to dendritic cells in vitro may be caused by increased sensitivity of bone marrow cells to GM-CSF signal. Consistent with this observation, the ratio of dendritic cells in vivo was normal in a young mouse under physiological condition, but increased over time or by immunization.

As described above, the invention provides a DCIR−/− mouse and indicates involvement of DCIR in autoimmune sialadenitis and arthritis.

The invention demonstrates that aged DCIR−/− mouse spontaneously develops characteristic autoimmune disease. DCIR−/− mice at 12 months of age showed increased serum immune globulin level, as well as elevated production of autoantibodies such as antinuclear antibody, rheumatoid factor and anti IIC antibody, compared to WT mice. Histologically, pathological change was observed in salivary gland and insertion of a DCIR−/− mouse. Infiltration of lymphocyte into salivary gland is a typical sign of Sjogren syndrome (SS), a representative chronic autoimmune disease affecting salivary gland. Several mouse strains such as NOD, MRL/Ipr and NZB/W F1 are widely accepted as SS model animal. Human patients with other autoimmune diseases including RA, mixed connective tissue disease (MCTD), ankylosing spondylitis (AS) and spondylarthritis (SpA) develop localized sialadenitis at high incidence, the development of which strongly correlates to production of rheumatoid factor. These findings indicate that DCIR plays an important role in stable function of immune system and that deficiency thereof induces autoimmune diseases.

Aged DCIR−/− mice also spontaneously develop delayed ankylosing arthropathy. Histopathology of these mice revealed erosive destruction of bone and insertion associated with infiltration of inflammatory cells.

However, histopathology of DCIR−/− mouse tissue was evidently different from that of other types of RA model mice such as a HTLV-I-Tg mouse, a IL-1 Ra-KO mouse and a CIA mouse. Unlike other model mice that develop characteristic synovitis similar to human RA, enthesitis was the primary abnormality in the DCIR−/− mouse and synovitis was rarely observed. Synovitis is a characteristic sign of RA, while enthesitis is characteristic in inflammatory rheumatoid diseases such as AS, SpA and psoriatic arthritis (PsA). Therefore, DCIR deficiency may induce characteristic inflammatory arthritis similar to AS, SpA and PsA.

The invention indicates that CIA is aggravated in a DCIR−/− mouse. After immunization with 11 type collagen, serum level of antigen specific IgG1 and IgG3 was markedly increased in a DCIR−/− mouse. Also, elevated antigen specific T cells response and immune response to IIC were observed in a DCIR−/− mouse. The ratio of dendritic cells in these mice was markedly higher and they were activated. Increased ratio of dendritic cells was observed in an aged DCIR−/− mouse, but not in a young DCIR−/− mouse under physiological condition. Dendritic cells present antigen on the cell surface and produce a variety of immune regulating cytokines, playing an important role in activation of T cells. The findings of the invention indicate that elevated function of dendritic cells in a DCIR−/− mouse triggers autoimmune.

Since dendritic cells differentiate from bone marrow stem cells by GM-CSF, GM-CSF produced by stimulation of IIC/CFA may be involved in proliferation of DC in CIA. The invention demonstrates that the elevated sensitivity of GM-CSF signal transduction induced by DCIR deficiency facilitates proliferation and differentiation of bone marrow derived dendritic cells in vitro (FIG. 10). Elevated phosphorylation of STAT5 induced by GM-CSF stimulation in DCIR defective bone marrow cells indicates inhibitory regulation of DCIR over GM-CSF signal transduction. It is reported that human DCIR recruits SHP-1 and SHP-2. SHP-1 is tyrosine phosphatase containing SH2 domains and negatively controls cytokine signal transduction including GM-CSF. Furthermore, a motheaten mouse defective in SHP-1 develops systemic autoimmune and severe inflammation. Since ITIM of mouse DCIR is functional and its amino acid sequence is identical to that of ITIM of human DCIR, it can be inferred that mouse DCIR recruits SHP-1 and SHP-2 to regulate GM-CSF signal transduction.

It was demonstrated that the production of IIC specific antibodies such as IgG1 and IgG3 subclasses was elevated in a DCIR−/− mouse with CIA. It is reported that in a DBA/1 strain mouse, collagen specific IgG2a class antibody is involved in development of CIA, but the level of antigen specific IgG2a antibody (129×B6) did not significantly increase in a DCIR−/− mouse with (129×B6) F1 hybrid background. A DCIR−/− mouse showed significantly increased production of IL-4 and IL-10 which are known to be involved in class switch of IgG1 and IgG3. Based on these findings, it is indicated that DCIR is involved in regulation of Th2 response.

Dendritic cells are involved in differentiation of Th1 and Th2 effector T cells. Dendritic cells exposed to intracellular parasitic pathogen facilitate Th1 response, and some kinds of parasites facilitate differentiation of Th2 cells by dendritic cells. It is known that differentiation of regulating T cells (Treg) also is regulated by dendritic cells, and that specific pathogen induces production of Treg. Therefore, interaction between dendritic cells and pathogen may play an important role to induce specific subset of effector T cells, and DCIR may be one of receptors on dendritic cells involving in differentiation of specific T cells.

Recent exhaustive genome-wide linkage analyses have revealed many regions sensitive to autoimmune diseases. Particularly, mouse 6F2 site band which has DCIR gene, and rat 4q42 and human 12p13 region on same chromosome relate to several inflammatory diseases such as arthritis, systemic lupus erythematosus (SLE), spontaneous diabetes mellitus, atherosclerosis, encephalomyelitis, asthma, respiratory tract hypersensitivity and allergy. DCIR deficiency triggers autoimmune-like disease. Thus, it is indicated that DCIR may be one of sensitivity genes of inflammatory diseases.

### Example 11

### Change in Bone Metabolism in DCIR−/− Mouse

In order to investigate role of DCIR in bone metabolism, X-ray, observation of tissue section and pQCT analysis were conducted using a DCIR−/− mouse at 12 months of age.

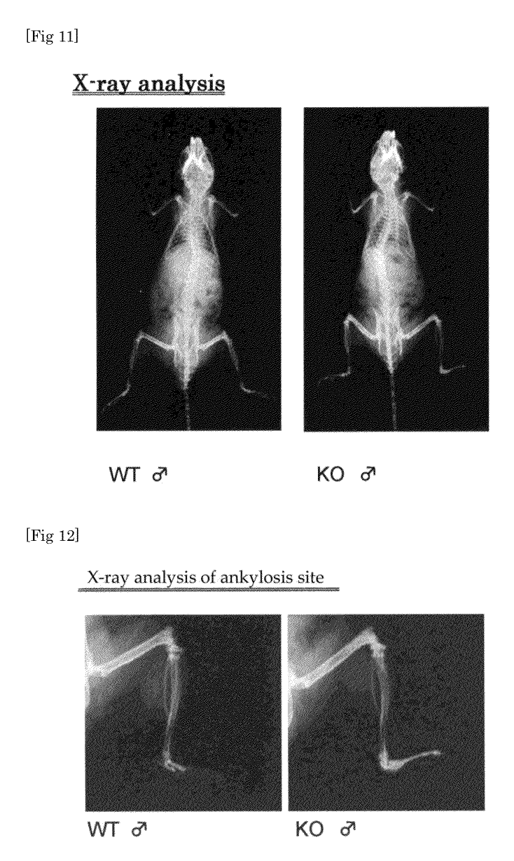

In a DCIR−/− mouse, only male mouse develops ankylosis at heel joint. X-ray of a DCIR−/− mouse and a WT mouse showed no difference in their skeleton, as shown in FIG. 11, demonstrating no problem in formation of skeleton.

On the other hand, X-ray revealed abnormality in the site of ankylosis (heel) developed in a DCIR−/− mouse. As shown in FIG. 12, the mouse had destroyed joint and increased calcification region at heel joint.

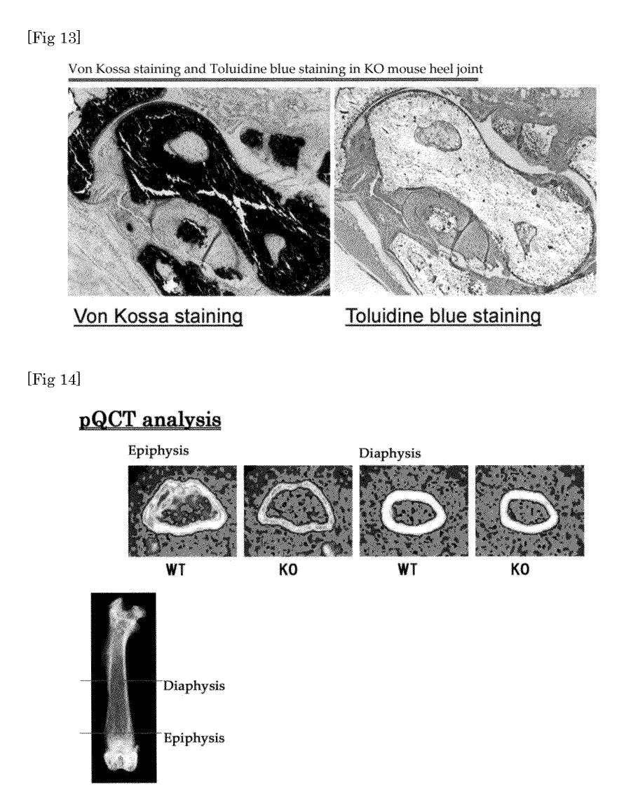

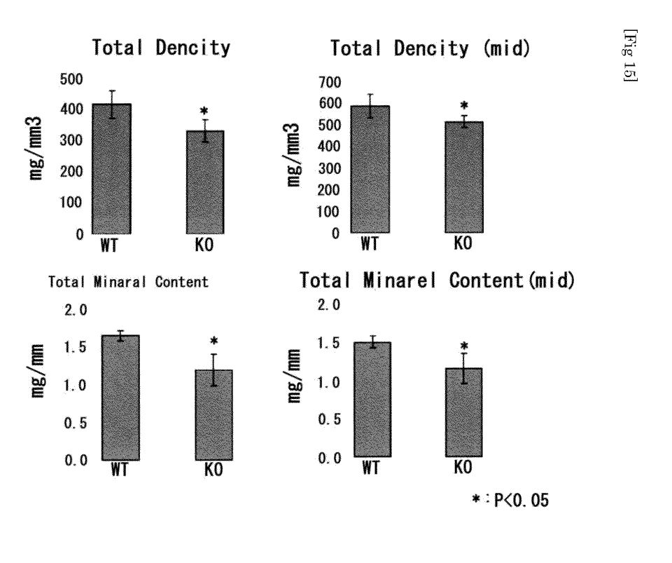

In order to further characterize the calcification region of the heel joint of a DCIR−/− mouse (KO), Von Kossa staining and Toluidine blue staining was used to investigate its cartilage tissue. As a result, proliferation of joint cartilage was observed in heel joint, with the cartilage replaced with bone (FIG. 13).

Next, in order to detect any abnormality in bone mass of femur, pQCT was conducted on an animal at 12 months of age (FIG. 14). The results were decreased bone density and bone mineral, that is, decreased bone mass (FIG. 15).

In summary, effects of DCIR gene include increased cartilage cells and ossified and calcified region in heel joint, and decreased bone mass in femur. It was indicated that DCIR may play a role in bone metabolism, regulation of differentiation and proliferation of cartilage cells, osteoblast and osteoclast cells. Based on these findings, a DCIR−/− mouse can serve as a good model mouse of osteoporosis, as well as AS and DISH.

Determination of Antitumor Immune Effect in DCIR−/− Mice:

### (1) Evaluation of Antitumor Immune Effect in Transplanted Cancer Experiment

Possible improvement in antitumor immune effect by DCIR deficiency as an inhibitory receptor was investigated using transplantable tumor cells.

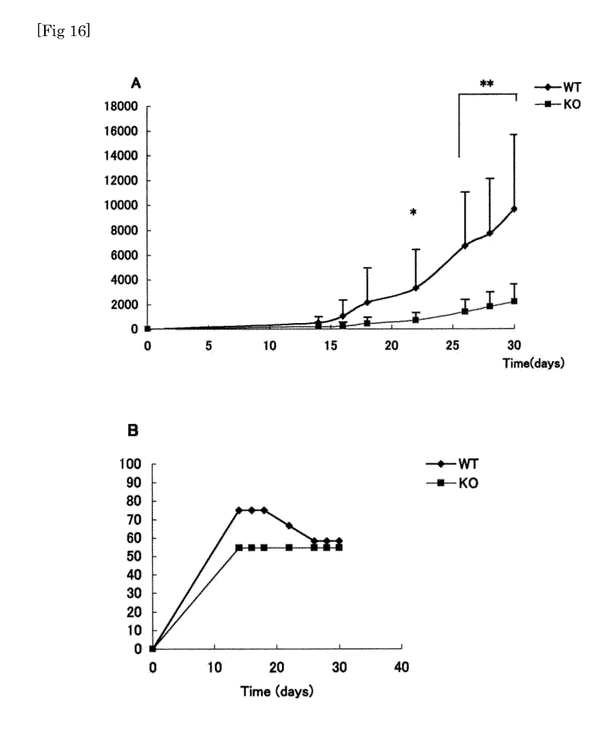

First, MethA was used as transplantable tumor cells. MethA was transplanted at dose of 1×10⁶ cell/head in DCIR−/− mice and WT mice, and difference in tumor formation was observed. There was no significant difference in incidence, but tumor mass was significantly lower in DCIR−/− mice than in WT mice (FIG. 16).

Next, BALB/3T3 APR-MUC1 (BALB/c background) which human MUC1 known as cancer antigen was expressed on BALB/3T3 as fibrosarcoma was used as transplantable tumor cells. BALB/3T3 APR-MUC1 clone16 supplied from Institute of Development, Aging and Cancer, Tohoku University was cultured in RPM1-1640(SIGMA) containing 10% FBS (SIGMA), 50U penicillin (Meiji Seika Kaisha Ltd.), 50 mg/ml streptomycin (Meiji Seika), and 500 mg/ml G418 (Nacalai Tesque), in subculture method at frequency of 2 to 3 a week.

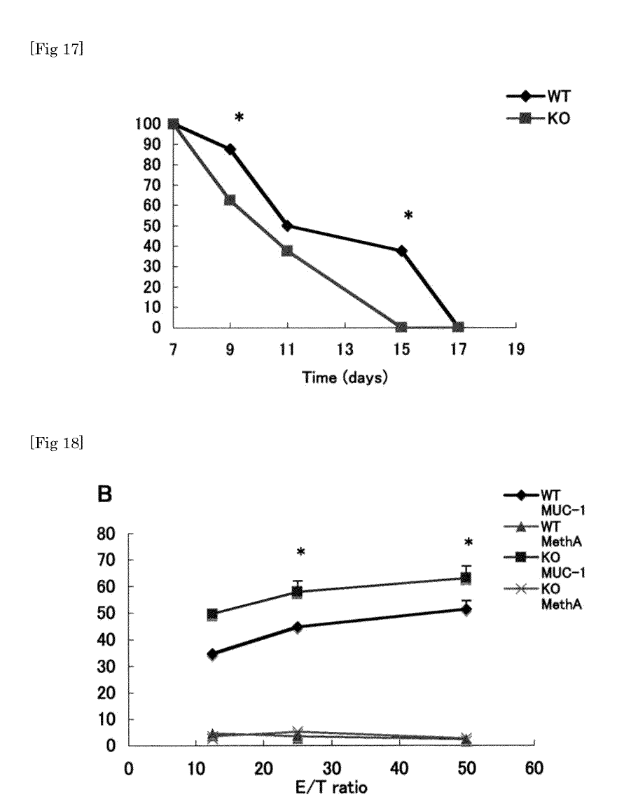

BALB-3T3 APR-MUC1 clone 16 was injected at dose of 1×10⁶ cell/head, and a palpable tumor (about 4 mm) was formed. Anti tumor immune effect was evaluated in terms of this incidence of tumor, and DCIR−/− mice showed significant reduction of tumor (FIG. 17).

### (2) Evaluation of Cytotoxic Activity of Dendritic Cells in DCIR−/− Mice

Induction of cytotoxic T cells (CTL) using tumor cell strain BALB/3T3 APR-MUC1 clone 16 was conducted as follows. Tumor cells were inactivated by 8000 rad γ-ray irradiation, and dosed intraperitoneally in mice at dose of 1×10⁷ cells/head at day 1 and 10. The spleen of the mice was collected 5 days after immunization to prepare spleen cells. The spleen cells were co-cultured with inactivated tumor cells on a 10-well plate in a ratio of 1×10⁷:1×10⁶, and stimulation was repeated for 3 days.

Target cells were labeled with 100μCi sodium chromate, ⁵¹Cr (GE Healthcare bioscience) at 37° C. for 1 hour, and suspended in RPMI-1640 (10% FBS, 10 mM 2-Mercaptoethanol). Lymphocytes were isolated from re-stimulated spleen cells by Lymphocyte-M (CEDARLANE), and co-cultured with the labeled target cells on a 96-well round-bottom plate (IWAKI). After 4 hours, emitted ⁵¹Cr was detected as radioactivity by Micro Bete (Pharmacia) to determine cytotoxic activity.

Cytotoxic activity was determined using BALB/3T3 APR-MUC1 clone16 to find out elevated activity in DCIR−/− mice. Damage to MethA as a negative control was not observed (FIG. 18). The finding demonstrates significant elevation in specific cytotoxic activity in DCIR−/− mice.

1. A nonhuman animal characterized by having a partial or complete deficiency of a chromosomal gene encoding a dendritic cell immune receptor (DCIR) protein.

2. The nonhuman animal of claim 1 wherein the animal is a rodent.

3. The nonhuman animal of claim 2 wherein the rodent is a mouse.

4. The nonhuman animal of claim 1 wherein the nonhuman animal is a model of an autoimmune disease.

5. The nonhuman animal of claim 4 wherein the autoimmune disease is rheumatoid arthritis (RA), ankylosing spondylitis (AS), psoriatic arthritis (PsA), Sjogren syndrome (SS), or diffused idiopathic skeletal hyperostosis (DISH).

6. The nonhuman animal of claim 1 wherein the nonhuman animal is a model of osteoporosis.

7. A method of screening a substance that facilitates or suppresses differentiation and proliferation of bone marrow stem cell derived cells, comprising:
administering a test substance to the nonhuman animal of claim 1, or to cells, tissues or organs derived from the animal, and
evaluating differentiation and proliferation of bone marrow stem cell derived cells in the animal, or cells, tissues or organs.

8. A method of screening a substance that suppresses development of arthritis, arthropathy or osteoporosis comprising:
administering a test substance to the nonhuman animal of claim 1, and
evaluating the incidence and severity of arthritis, arthropathy or osteoporosis in the animal.

9. The method of screening of claim 8 comprising:
comparing the result of said evaluating to the incidence and severity of arthritis, arthropathy or osteoporosis in wild type animal of identical species.

10. A method for treating an autoimmune disease or osteoporosis in a subject comprising:
identifying a substance using the screening method of claim 8, and
administering the substance to the subject.

11. A method for diagnosing an autoimmune disease or osteoporosis comprising:
administering a partial or complete gene coding dendritic cell immune receptor (DCIR) protein to a subject, and
evaluating arthritis, arthropathy or osteoporosis in the subject, wherein said evaluating provides a diagnosis of an autoimmune disease or osteoporosis.

12. The method of claim 11 wherein the autoimmune disease is rheumatoid arthritis (RA), ankylosing spondylitis (AS), psoriatic arthritis (PsA), Sjogren syndrome (SS), or diffused idiopathic skeletal hyperostosis (DISH).

13. A kit to screen a candidate substance that prevents autoimmune disease or osteoporosis comprising a peptide having an amino acid sequence identical or substantially identical to dendritic cell immune receptor (DCIR) protein, a partial peptide, or a salt thereof, or a candidate substance.

14. A kit to diagnose autoimmune disease or osteoporosis comprising DNA having a partial or entire base sequence identical or substantially identical to a gene coding dendritic cells immune receptor (DCIR) protein.

15. A method for treating a viral infection, a bacterial infection, protozoan infection, or a fungal infection in a subject, comprising:
administering a peptide having an amino acid sequence identical or substantially identical to dendritic cell immune receptor (DCIR) protein, a partial peptide or a salt thereof to a subject having a viral infection, a bacterial infection, protozoan infection, or a fungal infection.

16. A method for treating cancer in a subject, comprising:
administering an activating agent for cancer immunotherapy comprising a peptide having an amino acid sequence identical or substantially identical to dendritic cell immune receptor (DCIR) protein, a partial peptide or a salt thereof to a subject with cancer.

17. The method of screening of claim 7 comprising:
comparing the result of said evaluating to differentiation and proliferation of bone marrow stem cell derived cells in a wild-type animal of identical species, or in cells, tissues or organs derived from a wild-type animal of identical species.


SEQUENCE LISTING (17 sequences)

SEQ ID NO: 1 - DNA, 2404 bp, Mus musculus
ggcatttcccttatctcgccctggtgattctatgctgtggtttcttgttctcatctcgtttatcctagtgagacatgtctcttctttcatacaactgtgcaatatgacaacttatcacagtgattggttctcatatactatagagccttagagaaggaacaaggctctcttctgacggaggaagattttttcttgatatggcttcagaaatcacttatgcagaagtgaagttcaagaatgaatccaactccttgcacacctactcagaatctcctgcagctcccagagagaaacctatccgtgatctaagaaagcctggttccccctcactgcttcttacatccctgatgctacttctcctgctgctggcaatcacattcttagttgcttttatcatttattttcaaaagtactctcaacttcttgaagaaaaaaaagctgcaaaaaatataatgcacaatgaattgaactgcacaaaaagtgtttcacccatggaagacaaagtctggagctgttgcccaaaggattggaggctatttggttcccactgctacttggttcccacagtttcttcatcagcatcttggaacaagagtgaggagaactgctcccgcatgggtgctcatctagtggtgatccaaagccaggaagagcaggatttcatcactgggatcttggacactcatgctgcttattttatagggttgtgggatacaggccatcggcaatggcaatgggttgatcagacaccatatgaagaaagtatcacattctggcacaatggtgagcccagcagtggcaatgaaaaatgtgctacaataatttaccgttggaagactggatggggctggaacgatatctcttgcagtcttaaacagaagtcagtttgtcagatgaagaaaataaacttatgaatcactcattcttcatgggcattcgattcattgttatccaaccattacacagacacctgggaaattctacaggttcacagaatttaagtgggcagcaaatggttatgcatacactggcccacatatatccttgtgcatttacccacctactctgtcataaaatgaactttcattgagaattttctatataccacagagtatacagagtcccttatggacacacatggaactttttgccatcttgtttactcatgccattgtatgataggttctcttgacctatctgtttctgtttctctgttgtttttttaatgtctttggatttattgacattaaattgagaagtaaaattataaatatttaagtgtctggattgatacacacagatatgtactatgaaatataattaaatatttactgtcaaataattgtccaaagaaggttaacaacaccttcctttacttaatattattgctatattgcatgtgtatgtgtgacagaaagagaaagaggagagagagagagagagagagagagagagagagagagagagagagagagagagagagagagagaatgtgtgtgtgataagaatacttatggccacttaaagaaaattctgataaataggatgttgctaacatttatcattttattttaccatggatttcatatttatgtctcttttatcagcagtttgttcccttcatcaacatgtcagagaccccagtaccccaccctttacaaccatgtattcttcactcaaattcctgagattatttgataaattattatattgtaatattttacagattatctcttcatttaatttctctaatgattcatgaaaagcagcattcccagctaccaacatgcagtgattattgttttgtgtgttgattaaatgatgtttgagaacaagactggtttatagtttagacttagataaatatagaatgaaaaataaggcccaacttgctgatgtagctataatgctcatctaaaaaatgtgaatttatatacttaatataaaatagaaatattaagtattctttattagccattatatgaaaaccatactaaaatgcctttcagtaagtaaaaggcagcatattagacacaagatactctgactaaattggcatagagaagaagaatatagacaattgctttcttcttttcaaacattttctgtagcaagccaagggtgacaaatggaaacccagacaaaacctaactgagccctgagctagctcagacatattcatgtaaccagtttcataagtcaatatgtgaaataaaactgcatcagcctctgaggcagcattcagaaaattgaacattactacttaagctaattttctctttaggaaaggaaagcaaggatagtattctctaatctggaattggtcattttaagcattcattttaccatgagtaaattttacacgattttactcaaacccaataaaatattctgctagcccttc

SEQ ID NO: 2 - DNA, 1291 bp, Homo sapience
ctgtgattctcactatactggtcctgaggaaagggcttctgtgaactgcggtttttagtttttattgtggttcttagttctcatgagacccctcttgaggatatgtgcctatctggtgcctctgctctccactagttgagtgaaaggaaggaggtaatttaccaccatgtttggttcctgtttataagatgttttaagaaagatttgaaacagattttctgaagaaagcagaagctctcttcccattatgacttcggaaatcacttatgctgaagtgaggttcaaaaatgaattcaagtcctcaggcatcaacacagcctcttctgcagcttccaaggagaggactgcccctcacaaaagtaataccggattccccaagctgctttgtgcctcactgttgatatttttcctgctattggcaatctcattctttattgcttttgtcattttctttcaaaaatattctcagcttcttgaaaaaaagactacaaaagagctggttcatacaacattggagtgtgtgaaaaaaaatatgcccgtggaagagacagcctggagctgttgcccaaagaattggaagtcatttagttccaactgctactttatttctactgaatcagcatcttggcaagacagtgagaaggactgtgctagaatggaggctcacctgctggtgataaacactcaagaagagcaggatttcatcttccagaatctgcaagaagaatctgcttattttgtggggctctcagatccagaaggtcagcgacattggcaatgggttgatcagacaccatacaatgaaagttccacattctggcatccacgtgagcccagtgatcccaatgagcgctgcgttgtgctaaattttcgtaaatcacccaaaagatggggctggaatgatgttaattgtcttggtcctcaaaggtcagtttgtgagatgatgaagatccacttatgaactgaacattctccatgaacaggtggttggattggtatctgtcattgtagggatagataataagctcttcttattcatgtgtaagggaggtccatagaatttaggtggtctgtcaactattctacttatgagagaattggtctgtacattgactgattcactttttcataaagtgagcatttattgagcattttttcatgtgccagagcctgtactggaggcccccattgtgcacacatggagagaacatgagtctctcttaatttttatctggttgctaaagaattatttaccaataaaattatatgatgtggtgtctcaaaaaaaaaa

SEQ ID NO: 3 - DNA, 12722 bp, Mus musculus
  source 1..12722: /chromosome=“6” Targeted sequence
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcagaagattaaaacatattgccagaattcgtttgagatcaggcagagcaattcagtcagaagtcaagagaagccagtttgaatcagtcaggtttgagcggagttaggccagaacagctgagttgaaccagccagtgagagttcagaaagaactagaaaggcttgggaatggctcttatgtcatcccctcatccgaggaaataaaaacatttacatagagcaactattagtaaactctaaccaacctccctgaatgaccaacaactactaacccccatctattggggacctaacatttatgcatcatctgaagaattctcagaattccaaatgtcacacaattgcagaatctatttgcagctggcaagatgatgccccttccagggcaagaggtaaatcatagttgttatggtctgatttcaacattcagaataactgtagccattttgttccatgcctgggagctattttatattgcttctgtaacaagcctaccagccattgacacagaaaagtgatttgcctcaaggacatgctgaccacataccctgttttgttctttatgtttggactgttctgcatgaggtttgctaatcttaagaaattcctcaaagtctttcttaggactccagaaatcaaaggtcaatatgaactattatgtctgcaatggcttgagtcagagttgatcaccagacagcctttcctgcacctacatatgggttcactgtggttttgaagttgacactgaacaatggtactaataaaccaagaagttatacttacaatactcatgctctgttatctcaatgttctgcaattcccctgtttaccacccatccaccactcacctcactttactcaggaccaatcagcttaaaggctagctgataatcctttggctactaaagtgtactgctccccttttgccttttaaacttttgaacctgtttatatatatatgtgtatatatatatattctaccctgagaacagacaagaactgcacttatgccttctagtcatttaatggtcctgattgttcagtattaaggtgtgcattcattattcttgcttaattgagatcaatgtttgcttcattttagtggcaattactggaacccaacaatggtcagcggctgcaaagcagcttgatatcccgacacctgtgattaaaacaaaaatctagtcttataatatttctgtgtttttaaaagaaaacaaaattcctcaatcctcactacatttcccactttctgttttaagccattaattatgttgtgtgtagcggggaattataaacttctgctcagttctaatctttattgggtttgattgttattcacagcttttggattcctatggaaatataaacaaatccatctccctaatgcaaaactttcactggtttccattctgatgttagcacatccttatacaggatgatttaattccagaatatatatatatgtgtgtgtgtgtatatatatacacacatatatatattacctatattgcaatgtttcttggctgttttttctttctgaaatggttcattcatttttttttgctgtagtaccttgagccccagaggacattttctgatggtaagaaattaccctatcttttttggacatacattaaattgggacagtggcaatgcttgccatatcacctgcacacccctaaaacttgaggtcttctttctgatttatattgaaaagtaccattgctcttagatatgctttgatcattaacatactttccacaattaatgcaccatgcattttgatattcgagacctctcactataacttgacctatgatattagcatggtacacattagaaccaatattggtcacatcccttatccactcttccatgggtgctgtcatgcctttactgatctcataacttgttttttgggggggggttcgagacagggtttctctgtgtagccctggctgtcctggaactcactctgtagatcaggctggcctcgaactcagaaatccacctacctctgcctcccgattggtaggattaaaggcatgcgccaccaacgcccggtgagctcataacttttatattcaacattcatatcttcaaatgcccaggtctctcaaaacctgccttgtttcagggtctaatacagccttgctctcagctgagattaatctttgttacaacagcaacaacaacaacaacaacaacaacaacaacaacaacaacaacaacaactaccagtgaaggcttctccagagccttgtacaatcttagtaaataatgtggacacttttccagggctcctcaattttgtcttaaggtcttaaaactgctaagcaacatcattctatagaaacatcatcaaacctgttcttatagatcagagtatcaaccttcacccagcaactgatcttttatgatattcattcccctagcactaattcactgttcaatatttgctgtttcttctgtctgccaggttagtcattgcaactgtggtctaacctctaatactgctgttaccaatcccttccagttttggggaataactctactttgagaacctcagttatttagaatttgttttgcataagtttagtgcattcttatgaaattatggcttctttaaaattcattagagctattacttctatgggataccatcctatcttctcataaccttgttcctcatcattacattagttgtataacttttgggaaagctgtggtggtctataaccctgggctgcccctccacccactctggcagcacagttggttaggaattaaccctttccctcaaaatggcctgtttcatcagattgtactcctgctttttcagcttttctctgacccatccaccctctggcacaatttctttcctcattcttcctgtgggaagtctccattctcttgctcagtcactgggcctgagccttttatgttctcccttttcaatgatccttgctctcttgtgcttccatctccacccacagtttttccagttgcacagccaactttacattcctttcagaaacaaaagaatagattgaccccttttgtttacaccgcctttttttttgtattttgtgccccctcagttcgagtcacagtttttctaactcctcagccagtttttcaagtcttttttgaaataaaatatatgaagaaggaaaagaaaaaataagagaaatccctctgggagtaaagtagagataccccagtagtagtagatgcaataaacgttttgctggcttcttaaatacccatatcccccccccccaacatttcttttatggcacttgaaatcaaaattttcattttatccttaaattttgagctacaggctgagttgccatttgttgttacattttgggttcaccttttcccaccatttatctctgctcctcatcatttcacccccatataactagataggagaaagaggagagatagaaagaagagaaagatagagatccttgaatctaatctcttgcttgtttcttctttgagcatgactactaataatctgaaaacccctccccctcacaaccactaaccaccaattctgcctatcggggctctagaatttatatactctctgaaaatttcccaggattccaaacatgacacaattgcagaaactttctgaaactggaataagcacaccgccaccagatcatgaggttaattatcgtcagctgctgtgggaagtctgaaacaagcacaggttgctgcacctgagattcagaatgaaaatatattcttatatttctatgttttttaaaagaagtaaaaatttcaaaattctcaccacaagcttccttgcagagggtagaccaggcatgggtaaagagtgtgcatggtggaaaactcactgtgggacgttactgtctattaaggtaacgagaatatccatgcctggttgtggatggcctgacattgggtgttagcagtcaagtaggatgaggagtgtatctatgcacagaaggcagaaatggtgtcactttcagagaatcgtgaggtgagaatgctgtctgcttggtgggatatctctgtgtcgctgcttggtaccttccagggtgttgagagtttctgcccatcaaggtgaccttggtggtgtgctaaaacccagggaggctgagtggagtcttcccagagatcagcaccaggaatgagagcacatccagcttaggggacataggcttgacctgaggtttggagtccacccagaaagtacaggcctctaagtgaagtgtgagcaacattgtgggaaagagatgggagcatccaagagattatcacttgggagtttgagatagagataatgagcatagatttgtttttactcatagatgtagattagaaatgccaaattttggaaataaaacgaaacctgtggctttacattaaaatgggggtaatagtgttaacccatggttttcagcatgtttggatataaaagcaaaagaaaaccagtaaatacaaactctctctctctctctctgtgtgtcttatgcatgcacatgttgtttgattgttatgtgtagtgtaacatggaagtagctagaggagattcaaagaagggaagtctttggaagaactgaaggctgagcatgtggctgtgagctggaagatgtctgtgggagtgactcaaatgattccatctccagcagctcaagtaagtgtcccctcttgtactctgtgatccattttattctctcaactattcccaacagtgcagtgaacatttaaagcatgagctgaggagggctgggagtaagggaagagaggcacaaggagaggagagagtccaaccactgctactatccagagctgtttcaggagctctgggcatgtccagtagcaaagtgagtggagagtccccatgcaggtagttatgaatctatggcaggaggcagagtgctcagaggagcaacttcagaagaaacagaatgtgaactttgccttccccacactggtggcagctgagaaaaaagtgggaatccacacaatggtgagggctaatggtccactacaatgtggtatcaaaattttcctgttcagtcaaaatcatcgttgtgccttaagagtttgcttatgttgtctggttttccagaaaccacatttgcatatagaagtgagtgaatttatgtgtgctgcagagtgttttacaacagggttctctaatagcatggttcctctttctcctccttcctctttctttccttccccctttgagttcccactttgcagtacttgcatatcttgctgagtgggtttgagggctacaattcttattttcttatgttaagaggttgcatttcccttatctcgccctggtgattctatgctgtggtttcttgttctcatctcgtttatcctagtgagacatgtctcttctttcatacaactgtgcaatatgacaacttatcacagtgattggttctcatatactatagagccttagagaaggaacaaggctctcttctgacggaggaagattttttcttgatgggctgcagggaattcgagcgactcgagataacttcgtataatgtatgctatacgaagttatacgcgttcggatttgagctcgggtcgaccaagaaaggcggagtcttcggtatctcgggtggcgtaggggtcgtacggacgataacagaagggttaggagggggaacgacaggacggggtggggtggggggtcttatcttactgtggatgagtctgttacgctacgttaaaggagtaaaataatcctttcctgtcaccctcaccgtggaaggtcccagttccttccgtgccccctccccgtttgttgtctaccgaccgttgatcttccgtgtcagctccgactagtcgctcgagatctcttaactaggggagtcttcttgagcagttcttccgctatcttccgctacgcgacgcttagccctcgccgctatggcatttcgtgctccttcgccagtcgggtaagcggcggttcgagaagtcgttatagtgcccatcggttgcgatacaggactatcgccaggcggtgtgggtcggccggtgtcagctacttaggtcttttcgccggtaaaaggtggtactataagccgttcgtccgtagcggtacccagtgctgctctaggagcggcagcccgtacgcgcggaactcggaccgcttgtcaagccgaccgcgctcggggactacgagaagcaggtctagtaggactagctgttctggccgaaggtaggctcatgcacgagcgagctacgctacaaagcgaaccaccagcttacccgtccatcggcctagttcgcatacgtcggcggcgtaacgtagtcggtactacctatgaaagagccgtcctcgttccactctactgtcctctaggacggggccgtgaagcgggttatcgtcggtcagggaagggcgaagtcactgttgcagctcgtgtcgacgcgttccttgcgggcagcaccggtcggtgctatcggcgcgacggagcaggacgtcaagtaagtcccgtggcctgtccagccagaactgtttttcttggcccgcggggacgcgactgtcggccttgtgccgccgtagtctcgtcggctaacagacaacacgggtcagtatcggcttatcggagaggtgggttcgccggcctcttggacgcacgttaggtagaacaagttaccggctagggtataaccgacgtccagctttccgggcctctactccttctcctcttgtcgcgccgtctgcacgcgaaaacttcgcacgctcttacggcccggaggcctcctggaagcccgcgggcggggcggggactcgggcggggactcgggcgggggcctgggtggggaagggtcggagactcgggtctttcgcttcctcgtttcgacgataaccggcgacggggtttccggatgggcgaaggtaacgagtcgccacgacaggtagacgtgctctgatcactctgcacgatgaaggtaaacagtgcaggacgtgctgcgctcgacgccccgcccccccttgaaggactgatcccctcctcatcttccaccgcgcttccccggtggtttcttgcctcggccaaccgcggatggccacctacaccttacacacgctccggtctccggtgaacacatcgcggttcacgggtcgccccgacgatttcgcgtacgaggtctgacggaacccttttcgcggaggggatgggccatcttaaggaaatccgactcgagataacttcgtataatgtatgctatacgaagttatacgcgttcgctcggtacccatcaagcttatctgcacacatcaatggaccatgtctgccttagatgaagagaggttccactctagcagttggcatttaatggaaatatgaagaagaaatattcttcatggaaaattaatacaagggattctattgtgacttcccctttgtccttaaatttaattattggggaaaaatggagtccactctcagtatagaaactaaatcacactcgtgagtgtctgtgtggggctgtctttattagactgggaggtcaaatctacattttctcattggcagcaaggtgtcaaactgtatgtgtatagggctccttcatctaatgtgagcttgtagacaagaaagagctaggaagaaattgtccaagaaggagttcttcagctaattcctagctgcttctgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtattacagatgatgtatcagaccaagtcccaggaagaacacagaactctgttctcaagaaatgtagaggaaagttaatgaatcagtgtcagctaaatataacggcaatctcttattatcccagctacttaggatgttagatagtaggacagtaagtgtggagctagcctgggctatgtaaaaagactatatctgatgaaaataaactggggtctgcaacattgtttgggaagtgttgtattgtgatagctttcctaaaataggtgacacctagaactgtgggtaccaagaatgtcacagagggatgaaactgaagcacgagtggctgaaatacacagtgctaagtgaagaagaaatgagatttccctaggtagacacaagcaggatgtagaacatgtgctgtgcacccatacaaaaccatttatttctcttaaaatgtgttctttaatcaagtattgttttctacttaataaatccatgtcaatgtaatgagctaatacatatacttatttatctcatgcttctatggtttggcaatttaggctgatctccactgagtggctcctcttgtcctagttgagcttaggcatgtgtatgtgtccaggcatctcagactctgaggaagaggaggccagctggaatgacgaacagtgtggctcaactttatatgagttcttaggagggcctgggctcatttacatggcagcttggagatttccaagactgaaggtggcaatgagccaaggctctcaaaaaatttaactggagagtacatcagagatcatttttcacattctcttgttcagctcttgcagaaaactttacttttttttttttaaatttaaattgggacagttgcaaaatcccattgcaatagtatgtggacatagaagttaaaaattgtggtcattttttttttttttgcaaaatatcaatgccatggagaactgctgtaccatttcaaagagagaattggtatagccacatttaggttatagatggtgctttttacaggaatctgaaaatagaatctgtagagcaaagagagaaatgagagaaataatttataacctgataatgagataagtgtcaggaaaacatgagatgaggcagctttgagatttaaagagtataggaccaagaaaatagatgtttgatgattattgagatctaggaattaagaggtccaagaaataaaatgtgattttaatagtttgatgatgtaaactcagcaatcatgaaattattcaaagaggtgtagaatatataagccatatgtttaagttggtagatgttgacttgagttttagacatactgagttgaacctaatatgattgcatttagtgaccataaaatcttatgcaaaagggagcagacaagcaataccatagcaatacatgaccttactcaggaagaatttatatgagaagaaatcataatgacagggccgtaggaagtgttaaattgaaggggtggatggaaggagaagagattattaaagacaataaaaagtgattgtcaaaaggtgaggcaaaaccaagacaggcagcgtctaaagacctaagggttaagagaactaagcttacatccaataacaagatggctaatgagtgccctgggatattactgagtatacatttcaaaactggtgtttgagaaatggaggaagcataagtgatggcataatgaggacaacaacataggcaggaaagtgaggaaatctagttctagagggggaggccagttagttttaataggacagaatgggagtgtggaacatggaggaataagtcagtctaaatgaagaaagtttaaggaagaaagcaatgccctgcagatacttgtggttttttttttttttctgatttcacattctaaaataacatataactactcagaattacttaactccttttcctcccaatgcaaaggtagttgacatccttaaatatgactatttatctctgattcatctcctggaagaattaacattttcatcttacattcagctttgaaggatatggttcactccctttagacgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 22 bp, Artificial sequence
  PCR primer
catttcccttatctcgccctgg

SEQ ID NO: 5 - DNA, 22 bp, Artificial sequence
  PCR primer
gcagcatgaatgtccaagatcc

SEQ ID NO: 6 - DNA, 38 bp, Artificial sequence
  PCR primer
gattaaaagcggccgccagaattcgtttgagatcaggc

SEQ ID NO: 7 - DNA, 29 bp, Artificial sequence
  PCR primer
ctggatccgtcagaagagagccttgttcc

SEQ ID NO: 8 - DNA, 29 bp, Artificial sequence
  PCR primer
ccatcgatgaagagaggttccactctagc

SEQ ID NO: 9 - DNA, 29 bp, Artificial sequence
  PCR primer
ttatcgatgtcaactacctttgcattggg

SEQ ID NO: 10 - DNA, 22 bp, Artificial sequence
  primer for amplification
taacactgagggaagatgctac

SEQ ID NO: 11 - DNA, 22 bp, Artificial sequence
  primer for amplification
tctcattctcactctcactctc

SEQ ID NO: 12 - DNA, 18 bp, Artificial sequence
  primer for amplification
agccatgataacagaccc

SEQ ID NO: 13 - DNA, 19 bp, Artificial sequence
  primer for amplification
tgatatggggtctggtacg

SEQ ID NO: 14 - DNA, 24 bp, Artificial sequence
  PCR primer
aagtgtcccctcttgtactctgtg

SEQ ID NO: 15 - DNA, 25 bp, Artificial sequence
  primer for amplification
caaaattctgtcaagcgtagagggg

SEQ ID NO: 16 - DNA, 26 bp, Artificial sequence
  primer for amplification
cattatacgaagttatctcgagtcgc

SEQ ID NO: 17 - DNA, 12818 bp, Mus musculus
  source 1..12818: /chromosome=“6” Targeted sequence
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcagaagattaaaacatattgccagaattcgtttgagatcaggcagagcaattcagtcagaagtcaagagaagccagtttgaatcagtcaggtttgagcggagttaggccagaacagctgagttgaaccagccagtgagagttcagaaagaactagaaaggcttgggaatggctcttatgtcatcccctcatccgaggaaataaaaacatttacatagagcaactattagtaaactctaaccaacctccctgaatgaccaacaactactaacccccatctattggggacctaacatttatgcatcatctgaagaattctcagaattccaaatgtcacacaattgcagaatctatttgcagctggcaagatgatgccccttccagggcaagaggtaaatcatagttgttatggtctgatttcaacattcagaataactgtagccattttgttccatgcctgggagctattttatattgcttctgtaacaagcctaccagccattgacacagaaaagtgatttgcctcaaggacatgctgaccacataccctgttttgttctttatgtttggactgttctgcatgaggtttgctaatcttaagaaattcctcaaagtctttcttaggactccagaaatcaaaggtcaatatgaactattatgtctgcaatggcttgagtcagagttgatcaccagacagcctttcctgcacctacatatgggttcactgtggttttgaagttgacactgaacaatggtactaataaaccaagaagttatacttacaatactcatgctctgttatctcaatgttctgcaattcccctgtttaccacccatccaccactcacctcactttactcaggaccaatcagcttaaaggctagctgataatcctttggctactaaagtgtactgctccccttttgccttttaaacttttgaacctgtttatatatatatgtgtatatatatatattctaccctgagaacagacaagaactgcacttatgccttctagtcatttaatggtcctgattgttcagtattaaggtgtgcattcattattcttgcttaattgagatcaatgtttgcttcattttagtggcaattactggaacccaacaatggtcagcggctgcaaagcagcttgatatcccgacacctgtgattaaaacaaaaatctagtcttataatatttctgtgtttttaaaagaaaacaaaattcctcaatcctcactacatttcccactttctgttttaagccattaattatgttgtgtgtagcggggaattataaacttctgctcagttctaatctttattgggtttgattgttattcacagcttttggattcctatggaaatataaacaaatccatctccctaatgcaaaactttcactggtttccattctgatgttagcacatccttatacaggatgatttaattccagaatatatatatatgtgtgtgtgtgtatatatatacacacatatatatattacctatattgcaatgtttcttggctgttttttctttctgaaatggttcattcatttttttttgctgtagtaccttgagccccagaggacattttctgatggtaagaaattaccctatcttttttggacatacattaaattgggacagtggcaatgcttgccatatcacctgcacacccctaaaacttgaggtcttctttctgatttatattgaaaagtaccattgctcttagatatgctttgatcattaacatactttccacaattaatgcaccatgcattttgatattcgagacctctcactataacttgacctatgatattagcatggtacacattagaaccaatattggtcacatcccttatccactcttccatgggtgctgtcatgcctttactgatctcataacttgttttttgggggggggttcgagacagggtttctctgtgtagccctggctgtcctggaactcactctgtagatcaggctggcctcgaactcagaaatccacctacctctgcctcccgattggtaggattaaaggcatgcgccaccaacgcccggtgagctcataacttttatattcaacattcatatcttcaaatgcccaggtctctcaaaacctgccttgtttcagggtctaatacagccttgctctcagctgagattaatctttgttacaacagcaacaacaacaacaacaacaacaacaacaacaacaacaacaacaacaactaccagtgaaggcttctccagagccttgtacaatcttagtaaataatgtggacacttttccagggctcctcaattttgtcttaaggtcttaaaactgctaagcaacatcattctatagaaacatcatcaaacctgttcttatagatcagagtatcaaccttcacccagcaactgatcttttatgatattcattcccctagcactaattcactgttcaatatttgctgtttcttctgtctgccaggttagtcattgcaactgtggtctaacctctaatactgctgttaccaatcccttccagttttggggaataactctactttgagaacctcagttatttagaatttgttttgcataagtttagtgcattcttatgaaattatggcttctttaaaattcattagagctattacttctatgggataccatcctatcttctcataaccttgttcctcatcattacattagttgtataacttttgggaaagctgtggtggtctataaccctgggctgcccctccacccactctggcagcacagttggttaggaattaaccctttccctcaaaatggcctgtttcatcagattgtactcctgctttttcagcttttctctgacccatccaccctctggcacaatttctttcctcattcttcctgtgggaagtctccattctcttgctcagtcactgggcctgagccttttatgttctcccttttcaatgatccttgctctcttgtgcttccatctccacccacagtttttccagttgcacagccaactttacattcctttcagaaacaaaagaatagattgaccccttttgtttacaccgcctttttttttgtattttgtgccccctcagttcgagtcacagtttttctaactcctcagccagtttttcaagtcttttttgaaataaaatatatgaagaaggaaaagaaaaaataagagaaatccctctgggagtaaagtagagataccccagtagtagtagatgcaataaacgttttgctggcttcttaaatacccatatcccccccccccaacatttcttttatggcacttgaaatcaaaattttcattttatccttaaattttgagctacaggctgagttgccatttgttgttacattttgggttcaccttttcccaccatttatctctgctcctcatcatttcacccccatataactagataggagaaagaggagagatagaaagaagagaaagatagagatccttgaatctaatctcttgcttgtttcttctttgagcatgactactaataatctgaaaacccctccccctcacaaccactaaccaccaattctgcctatcggggctctagaatttatatactctctgaaaatttcccaggattccaaacatgacacaattgcagaaactttctgaaactggaataagcacaccgccaccagatcatgaggttaattatcgtcagctgctgtgggaagtctgaaacaagcacaggttgctgcacctgagattcagaatgaaaatatattcttatatttctatgttttttaaaagaagtaaaaatttcaaaattctcaccacaagcttccttgcagagggtagaccaggcatgggtaaagagtgtgcatggtggaaaactcactgtgggacgttactgtctattaaggtaacgagaatatccatgcctggttgtggatggcctgacattgggtgttagcagtcaagtaggatgaggagtgtatctatgcacagaaggcagaaatggtgtcactttcagagaatcgtgaggtgagaatgctgtctgcttggtgggatatctctgtgtcgctgcttggtaccttccagggtgttgagagtttctgcccatcaaggtgaccttggtggtgtgctaaaacccagggaggctgagtggagtcttcccagagatcagcaccaggaatgagagcacatccagcttaggggacataggcttgacctgaggtttggagtccacccagaaagtacaggcctctaagtgaagtgtgagcaacattgtgggaaagagatgggagcatccaagagattatcacttgggagtttgagatagagataatgagcatagatttgtttttactcatagatgtagattagaaatgccaaattttggaaataaaacgaaacctgtggctttacattaaaatgggggtaatagtgttaacccatggttttcagcatgtttggatataaaagcaaaagaaaaccagtaaatacaaactctctctctctctctctgtgtgtcttatgcatgcacatgttgtttgattgttatgtgtagtgtaacatggaagtagctagaggagattcaaagaagggaagtctttggaagaactgaaggctgagcatgtggctgtgagctggaagatgtctgtgggagtgactcaaatgattccatctccagcagctcaagtaagtgtcccctcttgtactctgtgatccattttattctctcaactattcccaacagtgcagtgaacatttaaagcatgagctgaggagggctgggagtaagggaagagaggcacaaggagaggagagagtccaaccactgctactatccagagctgtttcaggagctctgggcatgtccagtagcaaagtgagtggagagtccccatgcaggtagttatgaatctatggcaggaggcagagtgctcagaggagcaacttcagaagaaacagaatgtgaactttgccttccccacactggtggcagctgagaaaaaagtgggaatccacacaatggtgagggctaatggtccactacaatgtggtatcaaaattttcctgttcagtcaaaatcatcgttgtgccttaagagtttgcttatgttgtctggttttccagaaaccacatttgcatatagaagtgagtgaatttatgtgtgctgcagagtgttttacaacagggttctctaatagcatggttcctctttctcctccttcctctttctttccttccccctttgagttcccactttgcagtacttgcatatcttgctgagtgggtttgagggctacaattcttattttcttatgttaagaggttgcatttcccttatctcgccctggtgattctatgctgtggtttcttgttctcatctcgtttatcctagtgagacatgtctcttctttcatacaactgtgcaatatgacaacttatcacagtgattggttctcatatactatagagccttagagaaggaacaaggctctcttctgacggaggaagattttttcttgatgggctgcagggaattcgagcgactcgagataacttcgtataatgtatgctatacgaagttatacgcgttcggatttgagctcgggtcgaccaagaaaggcggagtcttcggtatctcgggtggcgtaggggtcgtacggacgataacagaagggttaggagggggaacgacaggacggggtggggtggggggtcttatcttactgtggatgagtctgttacgctacgttaaaggagtaaaataatcctttcctgtcaccctcaccgtggaaggtcccagttccttccgtgccccctccccgtttgttgtctaccgaccgttgatcttccgtgtcagctccgactagtcgctcgagatctcttaactaggggagtcttcttgagcagttcttccgctatcttccgctacgcgacgcttagccctcgccgctatggcatttcgtgctccttcgccagtcgggtaagcggcggttcgagaagtcgttatagtgcccatcggttgcgatacaggactatcgccaggcggtgtgggtcggccggtgtcagctacttaggtcttttcgccggtaaaaggtggtactataagccgttcgtccgtagcggtacccagtgctgctctaggagcggcagcccgtacgcgcggaactcggaccgcttgtcaagccgaccgcgctcggggactacgagaagcaggtctagtaggactagctgttctggccgaaggtaggctcatgcacgagcgagctacgctacaaagcgaaccaccagcttacccgtccatcggcctagttcgcatacgtcggcggcgtaacgtagtcggtactacctatgaaagagccgtcctcgttccactctactgtcctctaggacggggccgtgaagcgggttatcgtcggtcagggaagggcgaagtcactgttgcagctcgtgtcgacgcgttccttgcgggcagcaccggtcggtgctatcggcgcgacggagcaggacgtcaagtaagtcccgtggcctgtccagccagaactgtttttcttggcccgcggggacgcgactgtcggccttgtgccgccgtagtctcgtcggctaacagacaacacgggtcagtatcggcttatcggagaggtgggttcgccggcctcttggacgcacgttaggtagaacaagttaccggctagggtataaccgacgtccagctttccgggcctctactccttctcctcttgtcgcgccgtctgcacgcgaaaacttcgcacgctcttacggcccggaggcctcctggaagcccgcgggcggggcggggactcgggcggggactcgggcgggggcctgggtggggaagggtcggagactcgggtctttcgcttcctcgtttcgacgataaccggcgacggggtttccggatgggcgaaggtaacgagtcgccacgacaggtagacgtgctctgatcactctgcacgatgaaggtaaacagtgcaggacgtgctgcgctcgacgccccgcccccccttgaaggactgatcccctcctcatcttccaccgcgcttccccggtggtttcttgcctcggccaaccgcggatggccacctacaccttacacacgctccggtctccggtgaacacatcgcggttcacgggtcgccccgacgatttcgcgtacgaggtctgacggaacccttttcgcggaggggatgggccatcttaaggaaatccgactcgagataacttcgtataatgtatgctatacgaagttatacgcgttcgctcggtacccatcaagcttatctgcacacatcaatggaccatgtctgccttagatgaagagaggttccactctagcagttggcatttaatggaaatatgaagaagaaatattcttcatggaaaattaatacaagggattctattgtgacttcccctttgtccttaaatttaattattggggaaaaatggagtccactctcagtatagaaactaaatcacactcgtgagtgtctgtgtggggctgtctttattagactgggaggtcaaatctacattttctcattggcagcaaggtgtcaaactgtatgtgtatagggctccttcatctaatgtgagcttgtagacaagaaagagctaggaagaaattgtccaagaaggagttcttcagctaattcctagctgcttctgtcttgtccagtattatgagaaatctaaaagcaacctgagcaaatgggtggatttctgttcatcaaagatttgagaatgtatgaaaaatttaaaaaaatgcatgaaaagtcacatatttaggatgctttactcatttatttgcacactcagaataaagtaatagtaataatatagtaatagcacacattaatgtattacagatgatgtatcagaccaagtcccaggaagaacacagaactctgttctcaagaaatgtagaggaaagttaatgaatcagtgtcagctaaatataacggcaatctcttattatcccagctacttaggatgttagatagtaggacagtaagtgtggagctagcctgggctatgtaaaaagactatatctgatgaaaataaactggggtctgcaacattgtttgggaagtgttgtattgtgatagctttcctaaaataggtgacacctagaactgtgggtaccaagaatgtcacagagggatgaaactgaagcacgagtggctgaaatacacagtgctaagtgaagaagaaatgagatttccctaggtagacacaagcaggatgtagaacatgtgctgtgcacccatacaaaaccatttatttctcttaaaatgtgttctttaatcaagtattgttttctacttaataaatccatgtcaatgtaatgagctaatacatatacttatttatctcatgcttctatggtttggcaatttaggctgatctccactgagtggctcctcttgtcctagttgagcttaggcatgtgtatgtgtccaggcatctcagactctgaggaagaggaggccagctggaatgacgaacagtgtggctcaactttatatgagttcttaggagggcctgggctcatttacatggcagcttggagatttccaagactgaaggtggcaatgagccaaggctctcaaaaaatttaactggagagtacatcagagatcatttttcacattctcttgttcagctcttgcagaaaactttacttttttttttttaaatttaaattgggacagttgcaaaatcccattgcaatagtatgtggacatagaagttaaaaattgtggtcattttttttttttttgcaaaatatcaatgccatggagaactgctgtaccatttcaaagagagaattggtatagccacatttaggttatagatggtgctttttacaggaatctgaaaatagaatctgtagagcaaagagagaaatgagagaaataatttataacctgataatgagataagtgtcaggaaaacatgagatgaggcagctttgagatttaaagagtataggaccaagaaaatagatgtttgatgattattgagatctaggaattaagaggtccaagaaataaaatgtgattttaatagtttgatgatgtaaactcagcaatcatgaaattattcaaagaggtgtagaatatataagccatatgtttaagttggtagatgttgacttgagttttagacatactgagttgaacctaatatgattgcatttagtgaccataaaatcttatgcaaaagggagcagacaagcaataccatagcaatacatgaccttactcaggaagaatttatatgagaagaaatcataatgacagggccgtaggaagtgttaaattgaaggggtggatggaaggagaagagattattaaagacaataaaaagtgattgtcaaaaggtgaggcaaaaccaagacaggcagcgtctaaagacctaagggttaagagaactaagcttacatccaataacaagatggctaatgagtgccctgggatattactgagtatacatttcaaaactggtgtttgagaaatggaggaagcataagtgatggcataatgaggacaacaacataggcaggaaagtgaggaaatctagttctagagggggaggccagttagttttaataggacagaatgggagtgtggaacatggaggaataagtcagtctaaatgaagaaagtttaaggaagaaagcaatgccctgcagatacttgtggttttttttttttttctgatttcacattctaaaataacatataactactcagaattacttaactccttttcctcccaatgcaaaggtagttgacatccttaaatatgactatttatctctgattcatctcctggaagaattaacattttcatcttacattcagctttgaaggatatggttcactccctttagacgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac